(19) United States
(12) Patent Application Publication
(10) Pub. No.: US 2018/0244518 A1
(43) Pub. Date: Aug. 30, 2018

(54) METHOD OF ASSEMBLING NANOSCALE AND MICROSCALE OBJECTS INTO THREE-DIMENSIONAL STRUCTURES

(71) Applicants: Peter MIRAGLLA, Hanover, MA (US); Andrew DINEEN, Cambridge, MA (US); David J. CARTER, Concord, MA (US); THE CHARLES STARK DRAPER LABORATORY, INC., Cambridge, MA (US)

(72) Inventors: Peter Miraglia, Hanover, MA (US); Andrew Dineen, Cambridge, MA (US); David J. Carter, Concord, MA (US)

(21) Appl. No.: 15/580,614

(22) PCT Filed: Jun. 8, 2016
(86) PCT No.: PCT/US16/36456

§ 371 Date: Dec. 7, 2017

Related U.S. Application Data
(60) Provisional application No. 62/172,315, filed on Jun. 8, 2015.

(51) Int. Cl. B81C 3/00; C12Q 1/6806; G03F 7/039; G03F 7/038; G03F 7/26; B82B 3/00

(52) U.S. Cl. CPC B81C 3/005; C12Q 1/6806; G03F 7/039; G03F 7/038; B81C 2203/032; B81C 3/001; B82B 3/0052; B82B 3/0047; B81C 2203/057; G03F 7/26

(57) ABSTRACT

A method of assembly of micro/nano-scale objects into lattice or truss structures.

### CROSS-REFERENCE TO RELATED APPLICATION

This application claims priority under 35 U.S.C. § 119(e) to U.S. Provisional Application Ser. No. 62/172,315 titled “METHOD OF ASSEMBLING NANOSCALE AND MICROSCALE OBJECTS,” filed Jun. 8, 2015, which is incorporated herein by reference in its entirety for all purposes.

### BACKGROUND

One goal of modern materials science involves the production of macro-scale structures from micro-scale elements having dimensions on the order of microns or nanometers. Such structures may be tailored to have novel mechanical, electrical, and optical properties that are unobtainable using conventional manufacturing techniques. Conventional micro-scale manufacturing processes, for example, as used in the semiconductor industry are incapable of producing macro-scale structures from micro-scale elements. For example, conventional semiconductor manufacturing equipment and processes are incapable of producing micro-scale elements having aspect ratios much greater than about 50:1 or about 100:1. Conventional additive manufacturing equipment and processes (often referred to as “3-D printing”) are incapable of producing objects having dimensions on the order of microns or nanometers and are incapable of quickly producing macro-scale structures from micro-scale elements.

### SUMMARY

In accordance with an aspect disclosed herein, there is provided a method of assembly of micro/nano-scale objects into a three dimensional structure. The method comprises forming a pattern of a first functional moiety on a surface of a substrate, contacting the surface of the substrate with a first liquid suspension including first micro/nano-scale feedstock elements functionalized with a second functional moiety, complimentary to the first functional moiety, on first portions of the first micro/nano-scale feedstock elements and functionalized with a third functional moiety on second portions of the first micro/nano-scale feedstock elements, aligning the first portions of the first micro/nano-scale feedstock elements in the first liquid suspension with the surface of the substrate, and facilitating bonding the second functional moieties to the first functional moieties to form a first mesostructure pattern of the first micro/nano-scale feedstock elements on the surface of the substrate. The method further comprises contacting the first mesostructure pattern of the first micro/nano-scale feedstock elements on the surface of the substrate with a second liquid suspension including micro/nano-scale linker feedstock elements functionalized with a fourth functional moiety, complimentary to the third functional moiety, on first portions of the micro/nano-scale linker feedstock elements and functionalized with a fifth functional moiety on second portions of the micro/nano-scale linker feedstock elements, aligning the first portions of the micro/nano-scale linker feedstock elements in the second liquid suspension with the second portions of a first group of the first micro/nano-scale feedstock elements, and facilitating bonding the fourth functional moieties to the third functional moieties to form a second mesostructure of micro/nano-scale objects on the surface of the substrate. The method further comprises contacting the second mesostructure pattern of micro/nano-scale feedstock elements on the surface of the substrate with a third liquid suspension including second micro/nano-scale feedstock elements functionalized with a sixth functional moiety, complimentary to the fifth functional moiety, on first portions of the second micro/nano-scale feedstock elements and functionalized with a seventh functional moiety, on second portions of the second micro/nano-scale feedstock elements, aligning the first portions of the second micro/nano-scale feedstock elements in the third liquid suspension with the second portions of a first group of the micro/nano-scale linker feedstock elements, aligning the second portions of the second micro/nano-scale feedstock elements in the third liquid suspension with the second portions of a second group of the micro/nano-scale linker feedstock elements, and facilitating bonding the sixth and seventh functional moieties to the fifth functional moieties to form the three-dimensional structure of micro/nano-scale objects on the surface of the substrate.

In some embodiments, facilitating bonding the fourth functional moieties to the third functional moieties and facilitating bonding the sixth and seventh functional moieties to the fifth functional moieties includes leaving some of at least one of the fourth, fifth, sixth, or seventh functional moieties unbonded.

In some embodiments, the method further comprises contacting the three dimensional structure of micro/nano-scale objects with a third liquid suspension including third micro/nano-scale feedstock elements, aligning and positioning first portions of the third micro/nano-scale feedstock elements in the third liquid suspension with third portions of the second micro/nano-scale feedstock elements, and facilitating bonding the first portions of third micro/nano-scale feedstock elements to the third portions of the second micro/nano-scale feedstock elements.

In some embodiments, the first portions of the third micro/nano-scale feedstock elements are bonded to the third portions of the second micro/nano-scale feedstock elements with complimentary click chemical groups.

In some embodiments, portions of micro/nano-scale feedstock elements are bonded to portions of other micro/nano-scale feedstock elements with complimentary click chemical groups.

In some embodiments, the first portions of third micro/nano-scale feedstock elements are bonded to the third portions of the second micro/nano-scale feedstock elements with complimentary DNA strands.

In some embodiments, portions of micro/nano-scale feedstock elements are bonded to portions of other micro/nano-scale feedstock elements with complimentary DNA strands.

In some embodiments, aligning and positioning the first portions of the third micro/nano-scale feedstock elements with the third portions of the second micro/nano-scale feedstock elements includes aligning and positioning the first portions of the third micro/nano-scale feedstock elements with the third portions of the second micro/nano-scale feedstock elements with an electric field to create electrophoretic and/or dielectrophoretic forces.

In some embodiments, aligning and positioning portions of micro/nano-scale feedstock elements with portions of other micro/nano-scale feedstock elements includes aligning and positioning the portions of the micro/nano-scale feedstock elements with the portions of the other micro/nano-scale feedstock elements with an electric field to create electrophoretic and/or dielectrophoretic forces.

In some embodiments, aligning and positioning the first portions of the third micro/nano-scale feedstock elements with the third portions of the second micro/nano-scale feedstock elements includes aligning and positioning the first portions of the third micro/nano-scale feedstock elements with the third portions of the second micro/nano-scale feedstock elements utilizing flow of fluid in the third liquid suspension.

In some embodiments, aligning and positioning portions of micro/nano-scale feedstock elements with portions of other micro/nano-scale feedstock elements includes aligning and positioning the portions of the micro/nano-scale feedstock elements with the portions of the other micro/nano-scale feedstock elements utilizing flow of fluid in the third liquid suspension.

In some embodiments, aligning and positioning the first portions of the third micro/nano-scale feedstock elements with the third portions of the second micro/nano-scale feedstock elements includes aligning and positioning the first portions of the third micro/nano-scale feedstock elements with the third portions of the second micro/nano-scale feedstock elements utilizing a magnetic field.

In some embodiments, aligning and positioning portions of micro/nano-scale feedstock elements with portions of other micro/nano-scale feedstock elements includes aligning and positioning the portions of the micro/nano-scale feedstock elements with the portions of the other micro/nano-scale feedstock elements utilizing a magnetic field.

In some embodiments, aligning and positioning the first portions of the third micro/nano-scale feedstock elements with the third portions of the second micro/nano-scale feedstock elements includes aligning and positioning the first portions of the third micro/nano-scale feedstock elements with the third portions of the second micro/nano-scale feedstock elements utilizing optical trapping.

In some embodiments, aligning and positioning portions of micro/nano-scale feedstock elements with portions of other micro/nano-scale feedstock elements includes aligning and positioning the portions of the micro/nano-scale feedstock elements with the portions of the other micro/nano-scale feedstock elements utilizing optical trapping.

In some embodiments, the method further comprises contacting the three dimensional structure of micro/nano-scale objects with a fourth liquid suspension including one or more of nanotubes, nanorods, and nanoparticles, aligning and positioning first portions of the one or more of nanotubes, nanorods, and nanoparticles in the fourth liquid suspension with second portions of the third micro/nano-scale feedstock elements, and bonding the one or more of nanotubes, nanorods, and nanoparticles to the second portions of the third micro/nano-scale feedstock elements with complimentary chemical groups, for example, complimentary click chemical groups and/or complimentary DNA strands.

In some embodiments, one or more of the first, second, or third micro/nano-scale feedstock elements include one or more of nanotubes, nanorods, or nanoparticles.

In some embodiments, the one or more of nanotubes, nanorods, and nanoparticles comprise one of carbon nanotubes, nanorods, and nanoparticles, boron nanotubes, nanorods, and nanoparticles, or combinations thereof.

In some embodiments, the one or more of nanotubes, nanorods, and nanoparticles are bonded to the second portions of the third micro/nano-scale feedstock elements with complimentary click chemical groups.

In some embodiments, the one or more of nanotubes, nanorods, and nanoparticles are bonded to portions of other micro/nano-scale feedstock elements with complimentary click chemical groups.

In some embodiments, the one or more of nanotubes, nanorods, and nanoparticles are bonded to the second portions of the third micro/nano-scale feedstock elements with complimentary DNA strands.

In some embodiments, the one or more of nanotubes, nanorods, and nanoparticles are bonded to portions of other micro/nano-scale feedstock elements with complimentary DNA strands.

In some embodiments, aligning and positioning the first portions of the one or more of carbon nanotubes, nanorods, and nanoparticles with the second portions of the third micro/nano-scale feedstock elements includes aligning and positioning the first portions of the one or more of nanotubes, nanorods, and nanoparticles with the second portions of the third micro/nano-scale feedstock elements with an electric field to create electrophoretic and/or dielectrophoretic forces.

In some embodiments, aligning and positioning the portions of the one or more of carbon nanotubes, nanorods, and nanoparticles with portions of other micro/nano-scale feedstock elements includes aligning and positioning the portions of the one or more of nanotubes, nanorods, and nanoparticles with the portions of the other micro/nano-scale feedstock elements with an electric field to create electrophoretic and/or dielectrophoretic forces.

In some embodiments, the method further comprises concurrently bonding at least two of i) the first portions of the first micro/nano-scale feedstock elements to the substrate, ii) the second portions of the first group of the first micro/nano-scale feedstock elements to the first portions of the second micro/nano-scale feedstock elements, iii) the second portions of the second group of the first micro/nano-scale feedstock elements to the second portions of the second micro/nano-scale feedstock elements, iv) the first portions of the third micro/nano-scale feedstock elements to the third portions of the second micro/nano-scale feedstock elements, and v) the one or more of nanotubes, nanorods, and nanoparticles to the second portions of the third micro/nano-scale feedstock elements or the one or more of nanotubes, nanorods, and nanoparticles to portions of other micro/nano-scale feedstock elements.

In some embodiments, the third functional moiety is the same as the first functional moiety.

In some embodiments, the fourth functional moiety is the same as the second functional moiety.

In some embodiments, the third functional moiety is the same as the second functional moiety.

In some embodiments, the fourth functional moiety is the same as the first functional moiety.

In some embodiments, facilitating bonding the second functional moieties to the first functional moieties includes initiating bonding between the second functional moieties and the first functional moieties by one of application of thermal energy to the second functional moieties and/or the first functional moieties, application of radiation to the second functional moieties and/or the first functional moieties, exposing the second functional moieties and/or the first functional moieties to a chemical catalyst and/or by changing a pH of the first fluid suspension to facilitate bonding between the first and second functional moieties.

In some embodiments, facilitating bonding between complimentary functional moieties includes initiating bonding between the complimentary functional moieties by one of application of thermal energy to the complimentary functional moieties, application of radiation to the complimentary functional moieties, exposing the complimentary functional moieties to a chemical catalyst and/or by changing a pH of a fluid suspension in which the complimentary functional moieties are immersed.

In some embodiments, the method further comprises bonding the first functional moiety with a linker molecule to an adhesion element bonded to the surface of the substrate to form the pattern of the first functional moiety on the surface of the substrate.

In some embodiments, the adhesion element comprises one or more of a metal, silicon, and silicon dioxide.

In some embodiments, the method further comprises bonding the second functional moiety with a linker molecule to an adhesion element bonded to the first portion of the first micro/nano-scale feedstock element.

In some embodiments, the method further comprises bonding functional moieties with linker molecules to adhesion elements bonded to portions of micro/nano-scale feedstock elements.

In some embodiments, the method further comprises facilitating bonding a plurality of the second micro/nano-scale feedstock elements to the second portions of each of the first micro/nano-scale feedstock elements.

In some embodiments, the method further comprises facilitating bonding a plurality of micro/nano-scale feedstock elements to portions of single other micro/nano-scale feedstock elements.

In some embodiments, the method further comprises facilitating bonding a plurality of the first micro/nano-scale feedstock elements to individual bonding sites including the first functional moiety on the surface of a substrate.

In some embodiments, facilitating bonding the second functional moieties to the first functional moieties includes facilitating bonding a first click chemical group to a complimentary click chemical group.

In some embodiments, facilitating bonding the second functional moieties to the first functional moieties includes facilitating bonding a first DNA strand to a complimentary DNA strand.

In some embodiments, the method further comprises bonding the first micro/nano-scale feedstock elements to the surface of the substrate with an additional bonding mechanism.

In some embodiments, the method further comprises forming at least one of the first, second, and third micro/nano-scale feedstock elements by sequential infiltration synthesis of a domain of a block copolymer.

In some embodiments, the method further comprises forming micro/nano-scale feedstock elements by sequential infiltration synthesis of a domain of a block copolymer.

In some embodiments, the method further comprises forming at least one of the first, second, or third micro/nano-scale feedstock elements by a method comprising depositing a liquid phase block copolymer into an area defined on or in an upper layer of a multi-layer substrate, annealing the block copolymer to facilitate separation of the block copolymer into multiple aligned polymer domains, removing one of the polymer domains, etching through a remaining polymer domain and into the upper layer of the multi-layer substrate, and obtaining the at least one of the first, second, or third micro/nano-scale feedstock elements by separating etched portions of the upper layer of the multi-layer substrate from a second layer of the multi-layer substrate.

In some embodiments, the method further comprises forming micro/nano-scale feedstock elements by a method comprising depositing a liquid phase block copolymer into an area defined on or in an upper layer of a multi-layer substrate, annealing the block copolymer to facilitate separation of the block copolymer into multiple aligned polymer domains, removing one of the polymer domains, etching through a remaining polymer domain and into the upper layer of the multi-layer substrate, and obtaining micro/nano-scale feedstock elements by separating etched portions of the upper layer of the multi-layer substrate from a second layer of the multi-layer substrate.

In some embodiments, the method further comprises forming at least one of the first, second, or third micro/nano-scale feedstock elements by a method comprising depositing a liquid phase block copolymer into an area defined on or in an upper layer of a multi-layer substrate, annealing the block copolymer to facilitate separation of the block copolymer into multiple aligned polymer domains, converting one of the polymer domains into an inorganic material using sequential infiltration synthesis, removing one of the polymer domains, etching through a second of the polymer domains and into the upper layer of the multi-layer substrate using the inorganic material as an etch mask, and obtaining the at least one of the first, second, or third micro/nano-scale feedstock elements by separating etched portions of the upper layer of the multi-layer substrate from a second layer of the multi-layer substrate.

In some embodiments, the method further comprises forming micro/nano-scale feedstock elements by a method comprising depositing a liquid phase block copolymer into an area defined on or in an upper layer of a multi-layer substrate, annealing the block copolymer to facilitate separation of the block copolymer into multiple aligned polymer domains, converting one of the polymer domains into an inorganic material using sequential infiltration synthesis, etching through a second of the polymer domains and into the upper layer of the multi-layer substrate using the inorganic material as an etch mask, and obtaining the micro/nano-scale feedstock elements by separating etched portions of the upper layer of the multi-layer substrate from a second layer of the multi-layer substrate.

In some embodiments, the method further comprises, prior to separating the etched portions of the upper layer of the multi-layer substrate from the second layer of the multi-layer substrate, depositing and patterning a layer of photoresist on the micro/nano-scale feedstock elements, patterning of the layer of photoresist exposing portions of the micro/nano-scale feedstock elements, defining lengths of the micro/nano-scale feedstock elements by etching through exposed portions of the micro/nano-scale feedstock elements, functionalizing exposed end portions of the micro/nano-scale feedstock elements while the micro/nano-scale feedstock elements are embedded in the photoresist, and removing the photoresist.

In some embodiments, forming at least one of the first, second, or third micro/nano-scale feedstock elements includes forming at least one of the first micro/nano-scale feedstock elements with at least one dimension smaller than about 50 nm.

In some embodiments, forming at least one of the first, second, or third micro/nano-scale feedstock elements includes forming at least one of the first micro/nano-scale feedstock elements with at least one dimension smaller than about 5 nm.

In some embodiments, forming micro/nano-scale feedstock elements includes forming micro/nano-scale feedstock elements with at least one dimension between about 5 nm and about 50 nm.

In some embodiments, the method further comprises functionalizing the first micro/nano-scale feedstock elements with the second functional moiety by a method including depositing a first bonding material on the second portions of the first micro/nano-scale feedstock elements, and exposing the first bonding material to a multifunctional click chemical including a chemical group having an affinity for the first bonding material.

In some embodiments, the method further comprises functionalizing micro/nano-scale feedstock elements with functional moieties by a method including depositing a first bonding material on portions of the micro/nano-scale feedstock elements, and exposing the first bonding material to a multifunctional click chemical including a chemical group having an affinity for the first bonding material.

In some embodiments, depositing the first bonding material on the second portions of the first micro/nano-scale feedstock elements includes depositing one of gold, silicon, and silicon dioxide on the second portions of the first micro/nano-scale feedstock elements.

In some embodiments, depositing the first bonding material on the portions of the micro/nano-scale feedstock elements includes depositing one of gold, silicon, and silicon dioxide on the portions of the micro/nano-scale feedstock elements.

In some embodiments, exposing the first bonding material to the multifunctional click chemical includes exposing the first bonding material to a chemical including the chemical group having the affinity for the first bonding material, an intermediate chemical group bonded to the chemical group having the affinity for the first bonding material and a further chemical group having an affinity for a second bonding material.

In some embodiments, the intermediate chemical group comprises a polymer chain.

In accordance with another aspect, there is provided a method of assembly of micro/nano-scale objects into a three dimensional lattice or truss structure. The method comprises forming a first liquid suspension including first micro/nano-scale feedstock elements functionalized with a first functional moiety on first portions of the first micro/nano-scale feedstock elements and linker elements including a second functional moiety, complimentary to the first functional moiety, on first portions of the linker elements and a third functional moiety on second portions of the linker elements, aligning the first portions of the first micro/nano-scale feedstock elements in the first liquid suspension with the first portions of the linker elements, facilitating bonding the second functional moieties to the first functional moieties to bond the linker elements to the first portions of the first micro/nano-scale feedstock elements, contacting the first micro/nano-scale feedstock elements and linker elements with a second liquid suspension including second micro/nano-scale feedstock elements functionalized with a fourth functional moiety, complimentary to the third functional moiety, on first portions of the second micro/nano-scale feedstock elements and on second portions of the second micro/nano-scale feedstock elements, aligning the first portions of the second micro/nano-scale feedstock elements in the second liquid suspension with the second portions of a first group of the linker elements, aligning the second portions of the second micro/nano-scale feedstock elements in the second liquid suspension with the second portions of a second group of the linker elements, and facilitating bonding the fourth functional moieties to the third functional moieties to form the three dimensional lattice or truss structure.

In some embodiments, the method further comprises contacting the three dimensional lattice or truss structure with a third liquid suspension including third micro/nano-scale feedstock elements functionalized with a fifth functional moiety, complimentary to a sixth functional moiety on a third portion of at least a portion of the linker elements, on first portions of the third micro/nano-scale feedstock elements, aligning the first portions of the third micro/nano-scale feedstock elements in the third liquid suspension with the third portions of the at least a portion of the linker elements, and facilitating bonding the fifth functional moieties to the sixth functional moieties.

In some embodiments, the method comprises aligning the first, second, and third micro/nano-scale feedstock elements into an auxetic truss structure.

In accordance with another aspect, there is provided a three dimensional lattice or truss structure of micro/nano-scale objects. The structure comprises a plurality of first micro/nano-scale feedstock elements having first portions bonded to first portions of linker elements and a plurality of second micro/nano-scale feedstock elements having first portions bonded to second portions of the linker elements and second portions bonded to third portions of the linker elements.

In some embodiments, the first portions of the plurality of first micro/nano-scale feedstock elements are bonded to the first portions of the linker elements with click chemical bonds.

In some embodiments, at least a portion of one of the first micro/nano-scale feedstock elements and the second micro/nano-scale feedstock elements have length:width aspect ratios of at least about 20:1.

In some embodiments, the structure further comprises a plurality of the second micro/nano-scale feedstock elements bonded to each first micro/nano-scale feedstock element.

In some embodiments, the structure further comprises a plurality of third micro/nano-scale feedstock elements having first portions bonded to fourth portions of the linker elements.

In some embodiments, the first portions of the plurality of third micro/nano-scale feedstock elements are bonded to fourth portions of the linker elements with click chemical bonds.

In some embodiments, the first, second, and third micro/nano-scale feedstock elements are arranged into an auxetic truss.

In some embodiments, the structure further comprises a plurality of the third micro/nano-scale feedstock elements bonded to each second micro/nano-scale feedstock element.

In accordance with another aspect, there is provided a method of assembly of micro/nano-scale objects into a three dimensional structure. The method comprises forming a pattern of a first functional moiety on a surface of a substrate, contacting the surface of the substrate with a first liquid suspension including first micro/nano-scale feedstock elements functionalized with a second functional moiety, complimentary to the first functional moiety, on first portions of the first micro/nano-scale feedstock elements and functionalized with a third functional moiety on second portions of the first micro/nano-scale feedstock elements, aligning the first portions of the first micro/nano-scale feedstock elements in the first liquid suspension with the surface of the substrate, facilitating bonding the second functional moieties to the first functional moieties to form a first mesostructure pattern of the first micro/nano-scale feedstock elements on the surface of the substrate, contacting the first mesostructure pattern of the first micro/nano-scale feedstock elements on the surface of the substrate with a second liquid suspension including second micro/nano-scale feedstock elements functionalized with a fourth functional moiety, complimentary to the third functional moiety, on first portions of the second micro/nano-scale feedstock elements and on second portions of the second micro/nano-scale feedstock elements, aligning the first portions of the second micro/nano-scale feedstock elements in the second liquid suspension with the second portions of a first group of the first micro/nano-scale feedstock elements, aligning the second portions of the second micro/nano-scale feedstock elements in the second liquid suspension with the second portions of a second group of the first micro/nano-scale feedstock elements, and facilitating bonding the fourth functional moieties to the third functional moieties to form the three dimensional structure of micro/nano-scale objects on the surface of the substrate.

In some embodiments, facilitating bonding the fourth functional moieties to the third functional moieties includes facilitating bonding the fourth functional moieties to linker elements and facilitating bonding of the linker elements to the third functional moieties.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A illustrates a substrate patterned with a first group of click chemical groups;

FIG. 1B illustrates a solution including a first plurality of micro-scale feedstock elements functionalized with click chemical groups complimentary to the click chemical groups patterned on the substrate and the substrate in contact with the solution;

FIG. 1C illustrates the first plurality of micro-scale feedstock elements being bonded to the substrate with the click chemical groups;

FIG. 1D illustrates a solution including a second plurality of micro-scale feedstock elements applied to the substrate with the bonded first plurality of micro-scale feedstock elements;

FIG. 1E illustrates the second plurality of micro-scale feedstock elements being bonded to the first plurality of micro-scale feedstock elements with click chemical groups;

FIG. 1F illustrates a structure formed from the substrate, first plurality of micro-scale feedstock elements, second plurality of micro-scale feedstock elements, and a third plurality of micro-scale feedstock elements bonded to the second plurality of micro-scale feedstock elements;

FIG. 2 is a flowchart of an embodiment of a method of forming the structure of FIG. 1F.

FIG. 3A illustrates a structure formed during performance of a method of forming a plurality of micro-scale feedstock elements;

FIG. 3B illustrates another structure formed during performance of the method of forming the plurality of micro-scale feedstock elements;

FIG. 3C illustrates another structure formed during performance of the method of forming the plurality of micro-scale feedstock elements;

FIG. 3D illustrates another structure formed during performance of the method of forming the plurality of micro-scale feedstock elements;

FIG. 3D′ illustrates another structure formed during performance of the method of forming the plurality of micro-scale feedstock elements;

FIG. 3E illustrates another structure formed during performance of the method of forming the plurality of micro-scale feedstock elements;

FIG. 3F illustrates another structure formed during performance of the method of forming the plurality of micro-scale feedstock elements;

FIG. 3G illustrates another structure formed during performance of the method of forming the plurality of micro-scale feedstock elements;

FIG. 3H illustrates another structure formed during performance of the method of forming the plurality of micro-scale feedstock elements;

FIG. 3I illustrates another structure formed during performance of the method of forming the plurality of micro-scale feedstock elements;

FIG. 3J illustrates another structure formed during performance of the method of forming the plurality of micro-scale feedstock elements;

FIG. 4 is a flowchart of an embodiment of a method for forming a plurality of micro-scale feedstock elements;

FIG. 5A is an elevational view of a structure used to form a mold for forming a plurality of micro-scale feedstock elements;

FIG. 5A′ is a plan view of the structure of FIG. 5A;

FIG. 5B is an elevational view of another structure used to form a mold for forming a plurality of micro-scale feedstock elements;

FIG. 5B′ is a plan view of the structure of FIG. 5B;

FIG. 5C illustrates a structure formed during performance of a method of forming a plurality of micro-scale feedstock elements;

FIG. 5D illustrates another structure formed during performance of the method of forming the plurality of micro-scale feedstock elements;

FIG. 5E illustrates another structure formed during performance of the method of forming the plurality of micro-scale feedstock elements;

FIG. 5F illustrates another structure formed during performance of the method of forming the plurality of micro-scale feedstock elements;

FIG. 5G illustrates another structure formed during performance of the method of forming the plurality of micro-scale feedstock elements;

FIG. 5H illustrates another structure formed during performance of the method of forming the plurality of micro-scale feedstock elements;

FIG. 6 is a flowchart of an embodiment of a method for forming a plurality of micro-scale feedstock elements;

FIG. 7A illustrates an act in a method of forming a micro-scale or nano-scale feedstock element using a block copolymer;

FIG. 7B illustrates another act in the method of forming a micro-scale or nano-scale feedstock element using a block copolymer;

FIG. 7C illustrates another act in the method of forming a micro-scale or nano-scale feedstock element using a block copolymer;

FIG. 7D illustrates another act in the method of forming a micro-scale or nano-scale feedstock element using a block copolymer;

FIG. 7E illustrates another act in the method of forming a micro-scale or nano-scale feedstock element using a block copolymer;

FIG. 7F illustrates another act in the method of forming a micro-scale or nano-scale feedstock element using a block copolymer;

FIG. 7G illustrates another act in another method of forming a micro-scale or nano-scale feedstock element using a block copolymer;

FIG. 7H illustrates another act in the other method of forming a micro-scale or nano-scale feedstock element using a block copolymer;

FIG. 7I illustrates another act in the other method of forming a micro-scale or nano-scale feedstock element using a block copolymer;

FIG. 8A illustrates an act in another method of forming a micro-scale or nano-scale feedstock element using a block copolymer;

FIG. 8B illustrates another act in the other method of forming a micro-scale or nano-scale feedstock element using a block copolymer;

FIG. 8C illustrates another act in the other method of forming a micro-scale or nano-scale feedstock element using a block copolymer;

FIG. 9 illustrates a block copolymer exhibiting separated aligned polymer domains;

FIG. 10 illustrates a solution of DNA functionalized micro-scale feedstock elements in a solution in contact with a substrate functionalized with complimentary DNA;

FIG. 11 illustrates an octet truss formed of micro-scale or nano-scale feedstock elements;

FIG. 12 is a chart of thermal resistivity v. yield strength of various classes of materials;

FIG. 13A illustrates an act in a method of forming a lattice or truss structure from micro-scale or nano-scale feedstock elements;

FIG. 13B illustrates another act in the method of forming a lattice or truss structure from micro-scale or nano-scale feedstock elements;

FIG. 13C illustrates another act in the method of forming a lattice or truss structure from micro-scale or nano-scale feedstock elements;

FIG. 13D illustrates another act in the method of forming a lattice or truss structure from micro-scale or nano-scale feedstock elements;

FIG. 14A illustrates an act in a method of forming an inductor structure from micro-scale or nano-scale feedstock elements;

FIG. 14B illustrates another act in the method of forming the inductor structure from micro-scale or nano-scale feedstock elements;

FIG. 14C illustrates another act in the method of forming the inductor structure from micro-scale or nano-scale feedstock elements;

FIG. 14D illustrates another act in the method of forming the inductor structure from micro-scale or nano-scale feedstock elements;

FIG. 14E illustrates the conductive portion of an inductor structure formed from micro-scale or nano-scale feedstock elements;

FIG. 15A illustrates an auxetic material lattice structure formed from micro-scale or nano-scale feedstock elements;

FIG. 15B illustrates another auxetic material lattice structure formed from micro-scale or nano-scale feedstock elements;

FIG. 15C illustrates another auxetic material lattice structure formed from micro-scale or nano-scale feedstock elements;

FIG. 15D illustrates another auxetic material lattice structure formed from micro-scale or nano-scale feedstock elements;

FIG. 15E illustrates another auxetic material lattice structure formed from micro-scale or nano-scale feedstock elements;

FIG. 16A illustrates another auxetic material lattice structure formed from micro-scale or nano-scale feedstock elements;

FIG. 16B illustrates another auxetic material lattice structure formed from micro-scale or nano-scale feedstock elements;

FIG. 16C illustrates another auxetic material lattice structure formed from micro-scale or nano-scale feedstock elements;

FIG. 16D illustrates another auxetic material lattice structure formed from micro-scale or nano-scale feedstock elements;

FIG. 16E illustrates another auxetic material lattice structure formed from micro-scale or nano-scale feedstock elements;

FIG. 16F illustrates another auxetic material lattice structure formed from micro-scale or nano-scale feedstock elements;

FIG. 17A illustrates another auxetic material structure formed from micro-scale or nano-scale feedstock elements;

FIG. 17B illustrates another auxetic material structure formed from micro-scale or nano-scale feedstock elements; and

FIG. 18 illustrates another auxetic material structure formed from micro-scale or nano-scale feedstock elements.

### DETAILED DESCRIPTION

Aspects and embodiments disclosed herein are not limited in application to the details of construction and the arrangement of components set forth in the following description or illustrated in the drawings. Aspects and embodiments disclosed herein are capable of being practiced or of being carried out in various ways. Also, the phraseology and terminology used herein is for the purpose of description and should not be regarded as limiting. The use of “including,” “comprising,” “having,” “containing,” “involving,” and variations thereof herein is meant to encompass the items listed thereafter and equivalents thereof as well as additional items.

Aspects and embodiments disclosed herein are generally directed to the formation of novel macro-scale structures from micro-scale or nano-scale elements having dimensions on the order of microns or nanometers. The disclosed macro-scale structures have mechanical, electrical, thermal, and/or optical properties that are unobtainable using conventional manufacturing techniques. Aspects and embodiments disclosed herein include the formation of macro-scale objects from micro-scale or nano-scale elements using a combination of directed fluidic assembly and “click” chemistry and/or DNA selective assembly techniques. Although the term “micro-scale elements” is used herein, it should be understood that the feedstock elements or other structures described herein are not limited to having dimensions of a micron or greater. The term “micro-scale elements” also encompasses feedstock elements or other structures having characteristic dimensions (length, width, etc.) smaller than one micron, for example, as small as less than about 1 nanometer. The term “micro-scale elements” or variations thereof is used synonymously with the term “micro/nano-scale elements” and variations thereof.

### Directed Fluidic Assembly (DFA)

Directed Fluidic Assembly (DFA) is an assembly method that allows structures made by dissimilar methods to be assembled together. It can be combined with planar micro/nanofabrication, micro-machining, 3D printing, and other fabrication modalities. DFA provides for rapid placement of homogeneous or heterogeneous feedstock onto a substrate or to other feedstock elements with controlled position and orientation. An advantage of DFA lies in the ability to use optimal methods to fabricate individual micro/nano components and assemble them into a permanently-bonded functional mechanical, electrical, thermal, fluidic, and/or thermal system. In some implementations DFA assembly is rapid: a feedstock spacing of 5 μm over a 100 mm wafer with a 2-minute assembly time corresponds to a rate of 2.5 million objects bonded per second. Smaller feedstocks will assemble at even higher rates.

Aspects and embodiments disclosed herein utilize a DFA technique for directed fluidic assembly of submicron to tens-of-micron-scale objects (feedstock) into millimeter-scale or larger structures (macro-scale structures). In some embodiments, high-aspect-ratio micro/nanofabricated feedstock structures of the same or of different length scales are fabricated in the plane of a substrate, released, and then combined by DFA into multiscale structures that have high aspect ratios perpendicular to a substrate or into three dimensional lattice or truss structures. In some embodiments, bonds to and between feedstock elements are permanent and provide for electrical conduction, thermal conduction, and/or optical transmission as required by the assembled system.

In some embodiments, DFA techniques for assembling micro-scale elements into larger composite structures include methods utilizing dielectrophoresis, electrophoresis, directed fluid flow, convection, capillary forces, magnetic fields, diffusion, optical trapping, or combinations thereof for orienting and positioning the micro-scale elements during fabrication. Many approaches have been used to assemble particles and other micro and nano building blocks onto conductive or insulating surfaces or structures. The control and speed of the assembly depends on many parameters, for example, particle size, concentration, charge, flow speed and direction, voltage, frequency, dielectric constant, etc. When using assembly mechanisms that depend on fluidic, capillary or other forces, the assembly forces, although controlled, can not be turned on and off (on demand) as in dielectrophoresis (DEP) or electrophoresis (EP) based assembly. Electrophoresis is a directed assembly method for fast assembly but it requires micro-scale elements to be oriented during a fabrication process to have a charge. DEP assembly forces depend only on the dielectric constant of the particle or the feedstock and therefore are more suitable for use to assemble uncharged feedstock. DEP assembly may be used to assemble nano and micro scale particles, rods or bars, and/or nanotube bundles into two and three-dimensional structures in seconds over a large area with precise alignment at desired locations. Based on the dilution of the feedstock solution and the strength of the applied electric field one can control the rate of assembly. Since the DEP force polarizes the feedstock, it leads to alignment of feedstock to orient the feedstock during assembly. Directionality of the nanomaterials as well as nanoscale feedstocks during assembly can effectively be controlled by controlling the applied electric field lines/gradients. The DEP assembly force can be effectively applied at the nano or microscale.

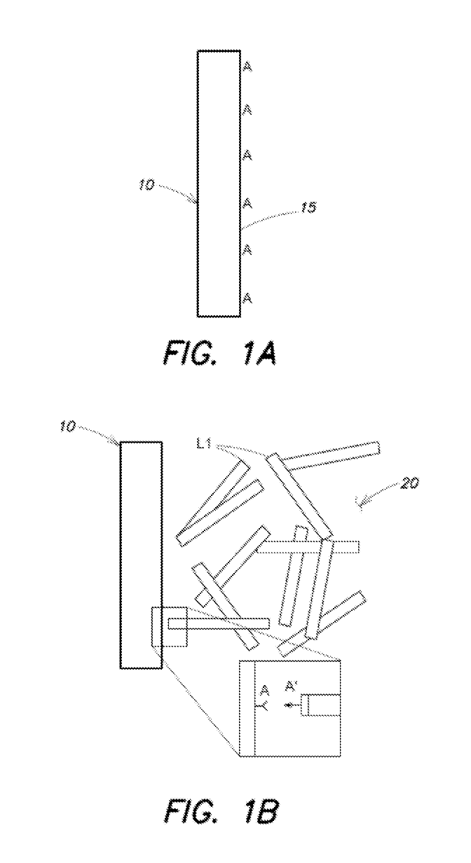

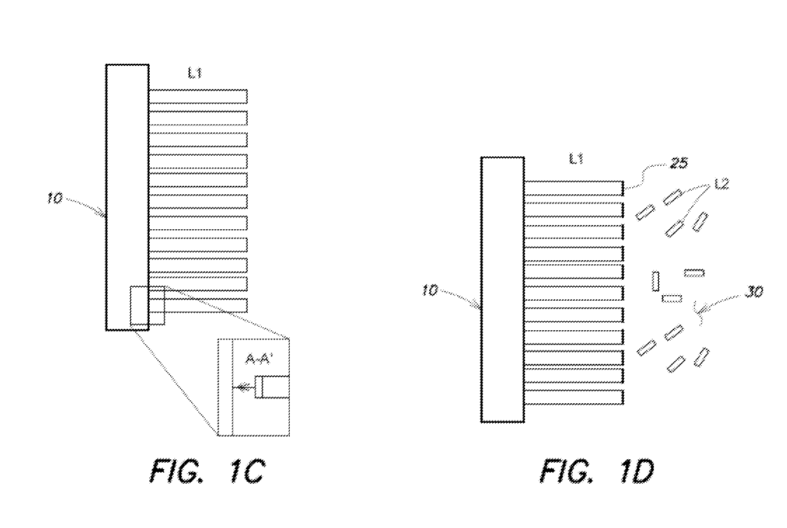

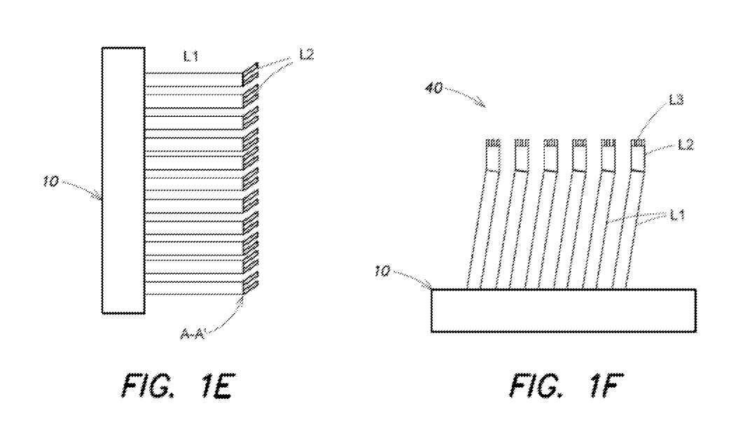

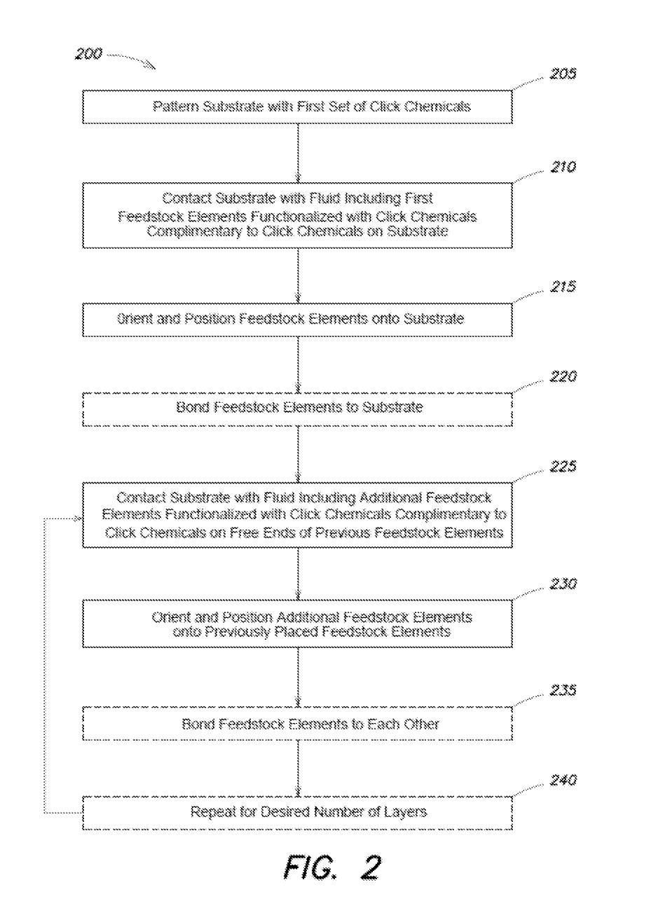

An embodiment of a DFA process 200 for fabricating an array of objects from two layers of micro-scale elements is shown schematically in FIGS. 1A-1E and is represented in the flowchart of FIG. 2. In act 205 of FIG. 2, represented in FIG. 1A, a substrate material 10, for example, a silicon wafer is patterned with a first set of functional moieties A, also referred to herein as “click chemicals.” The substrate 10 is patterned such that the functional moieties A are present in areas on the surface 15 of the substrate 10 where it is desired to connect micro-scale feedstock elements to the substrate 10. For example, the substrate 10 may be patterned with gold (Au) via electron-beam lithography and liftoff, or other patterning methods known in the art. A bifunctional molecule with one end being a thiol and the other end being an azide (the A side of the click reaction) may be placed in solution with the substrate. The thiol would then bind to the patterned gold surface, leaving the azide exposed for subsequent assembly to an alkyne (the A′ side of the click reaction)-functionalized feedstock in a subsequent step.

In act 210 of FIG. 2, represented in FIG. 1B, the patterned substrate 10 is placed in a fluid 20, for example, water, a buffer solution, an ionic liquid, or an organic solvent, containing micro-scale feedstock elements L1 that are functionalized with a click chemical A′ complimentary to the click chemical A present on the surface 15 of the substrate 10. As the term is used herein, “complimentary” chemical groups, moieties, or structures are chemical groups, moieties, or structures that bond specifically to one another. The micro-scale feedstock elements L1 may be in the form of micro-scale rods, bars, or cylinders having click chemical A′ present at one or both ends of the rods, bars, or cylinders. In one example, the feedstock elements L1 (or elements L2 or L3, referenced below) are fabricated laying on a surface (e.g., a silicon wafer) lined up in a two-dimensional array. The wafer may be placed in an electron-beam evaporator at a steep angle to the directional evaporation source such that one end of all the feedstock elements is exposed to the evaporated metal (e.g., gold) and a thin film of the metal is deposited only on those ends. The wafer is then turned 180 degrees and a metal (possibly gold, possibly another metal or dielectric) is deposited on the other end faces. The feedstock elements are then released from the substrate by etching away the underlying layer. The feedstock elements are placed in solution with a bifunctional molecule with one end being a thiol and the other end being an alkyne (the A′ side of the click reaction). The thiol will bind to the gold, leaving the alkyne exposed for subsequent assembly to an azide on a next feedstock element.

FIG. 1B illustrates a homogeneous population of micro-scale feedstock elements L1, however, in other embodiments, the fluid 20 may include a heterogeneous population of micro-scale feedstock elements of different sizes and/or shapes. In some embodiments, different click chemicals may be patterned on different areas of the substrate 10. Differently sized and/or shaped micro-scale feedstock elements in the fluid 20 may be provided with different click chemicals complimentary to the different click chemicals patterned on the substrate 10 so that the differently sized and/or shaped micro-scale feedstock elements may be bonded to different areas of the substrate 10 in a single process.

In act 215 of FIG. 2, represented in FIG. 1C, the micro-scale feedstock elements L1 are oriented and positioned on the surface 15 of the substrate 10. In different embodiments, any one or more of dielectrophoresis, electrophoresis, flow, convection, capillary forces, and magnetic fields, diffusion, or combinations thereof are used to orient and position the feedstock onto the substrate. In some embodiments, the substrate material 10 is conductive (for example, highly doped silicon) and/or is coated with a conductive thin film, for example, a metal, indium tin oxide, or another conductive material to facilitate the spread of charge across the surface of the substrate material 10 to aid in creating an electric field to align, orient, and/or move the micro-scale feedstock elements L1 into place on the surface 15 of the substrate 10. Once the micro-scale feedstock elements L1 are in position, the click chemistry locks the micro-scale feedstock elements L1 in place by forming a covalent bond between click chemicals A and A′. In other embodiments, described more fully below, complimentary DNA strands may be used instead of or in addition to complimentary click chemicals to bond the feedstock elements L1 to the surface 15 of the substrate 10 and/or to bond different feedstock elements to one another. In some embodiments, the covalent bond between click chemicals A and A′ is initiated by the addition of energy, for example, heat or ultraviolet light and/or a chemical initiator (act 220 of FIG. 2). It should be appreciated that areas on the substrate that are patterned with the functional moieties A may have areas significantly greater than the diameters of the feedstock elements L1, for example, areas on the substrate that are patterned with the functional moieties A may have areas on the order of from about 10 nm² to about 1 μm² while the diameters of the feedstock elements L1 may be on the order of from about 1 nm to about 50 nm. In other embodiments the areas on the substrate that are patterned with the functional moieties A may have areas of between about two and about 10 times greater than the cross sectional areas of the portions of the feedstock elements L1 that bond to the substrate. Thus, multiple feedstock elements L1 may be bond to multiple or to each area on the substrate that is patterned with the functional moieties A. Accordingly, illustration of individual feedstock elements in the figures should be considered as also representing multiple feedstock elements for each feedstock element illustrated.

In act 225 of FIG. 2, represented in FIG. 1D, the substrate 10 having the micro-scale feedstock elements L1 bonded thereto is contacted with a second liquid 30 including a second layer of micro-scale feedstock elements L2. The free ends 25 of the micro-scale feedstock elements L1 are functionalized with another click chemical that is complimentary to a click chemical present on ends of the micro-scale feedstock elements L2. In some embodiments, the free ends 25 of the micro-scale feedstock elements L1 are functionalized with the same click chemical A′ that the ends bonded to the substrate included and the micro-scale feedstock elements L2 are functionalized with the click chemical A that was patterned on the surface 15 of the substrate. In other embodiments, different click chemical pairs B—B′ are used to bond the first and second layers of micro-scale feedstock elements L1, L2. In some embodiments, liquid 30 is the same liquid as liquid 25 and bonding of the micro-scale feedstock elements L1 to the substrate 10 may occur concurrently with bonding of the micro-scale feedstock elements L2 to the micro-scale feedstock elements L1. In some embodiments, different triggers, for example, different types or levels of energy or different chemical initiators are used to initiate bonding of the micro-scale feedstock elements L1 to the substrate 10 and bonding of the micro-scale feedstock elements L2 to the micro-scale feedstock elements L1.

In act 230 of FIG. 2, represented in FIG. 1E, the micro-scale feedstock elements L2 are oriented and positioned on the micro-scale feedstock elements L1, for example, in an end-to-end configuration. In other embodiments, described in further detail below, the micro-scale feedstock elements L2 are oriented and positioned on the micro-scale feedstock elements L1 at an angle, for example, with their longitudinal axes oriented substantially perpendicular to the longitudinal axes of the micro-scale feedstock elements L1. In different embodiments, any one or more of DEP, diffusion, and/or convection are used to orient and position the micro-scale feedstock elements L2 on the micro-scale feedstock elements L1. Once the micro-scale feedstock elements L2 are in position, the click chemistry locks the micro-scale feedstock elements L2 in place on the micro-scale feedstock elements L1 by forming a covalent bond between click chemicals A and A′. In some embodiments, the covalent bond between click chemicals A and A′ is initiated by the addition of energy, for example, heat or ultraviolet light and/or a chemical initiator (act 235 of FIG. 2). In some embodiments, the functionalized areas of the feedstock elements L1 may be greater than the diameters of the feedstock elements L2 so that multiple feedstock elements L2 may bond to individual functionalized areas of the feedstock elements L1.

In accordance with process 200, additional layers of feedstock material may be added to previously bonded micro-scale feedstock elements until a desired number of layers is reached to form a desired macro-scale object (act 240 of FIG. 2). For example, a structure 40 including a substrate 10 and three layers of micro-scale feedstock elements L1, L2, and L3, is illustrated in FIG. 1F. In some embodiments, the one or more of the micro-scale feedstock elements L1, L2, and L3, or additional micro-scale feedstock elements connected directly or indirectly to elements L3, may be connected substantially perpendicular to the substrate 10 or at an angle of between zero degrees and about 45 degrees relative to the substrate. In some embodiments, the one or more of the micro-scale feedstock elements L1, L2, and L3, or additional micro-scale feedstock elements connected directly or indirectly to elements L3, may be connected substantially co-linearly to one or more other of the feedstock elements or at an angle of between zero degrees and about 45 degrees relative to one or more other of the feedstock elements. In some embodiments, the micro-scale feedstock elements L1 may be rods, bars, or cylinders having dimensions of about 100 micrometers (μm) by about 5 μm, micro-scale feedstock elements L2 may be rods, bars, or cylinders having dimensions of about 10 μm by about 0.5 μm, and micro-scale feedstock elements L3 may be rods, bars, or cylinders having dimensions of about 1 μm by about 0.1 μm. These dimensions are examples only and do not limit the present disclosure. Method 200 is not limited to only 3 layers of micro-scale feedstock elements; any number of layers of similarly or differently shaped and sized micro-scale feedstock elements may be connected to form a macro-structure as disclosed herein. In some embodiments micro-scale feedstock elements comprising or consisting of single or multi-walled carbon nanotubes or nanorods or nanoparticles of metals, polymers, or dielectrics, having lengths and/or widths of less than a micron may be utilized.

By patterning the substrate and faces or ends of the feedstock with click chemicals, 2-D and 3-D structures may be created. By patterning click chemicals on specific locations on the faces, ends, or portions of the sides of the feedstock, different layers of feedstock elements may be oriented at any desired orientation relative to each other. DFA is a rapid and scalable manufacturing technique due to its parallel nature. However, compared to some slower pick-and-place manufacturing techniques, DFA may suffer from defects, and thus may be best suited for defect-tolerant applications. For less defect tolerant structures, DFA could be combined with error-checking and/or pick-and-place correction techniques to achieve low defect levels at high fabrication rates.

### Fabrication of Micro-Scale Elements

Micro-scale feedstock elements utilized in forming 2-D and 3-D structures disclosed herein may be formed from materials including, for example, silicon, silicon dioxide, silicon nitride, silicon carbide, alumina, titania, zinc oxide, tungsten, SU-8 photoresist or other organic or inorganic polymers, biologically-based materials, for example, chitosan, or other materials selected based on, for example, desired mechanical, thermal, optical, electrical, magnetic, and/or chemical properties.

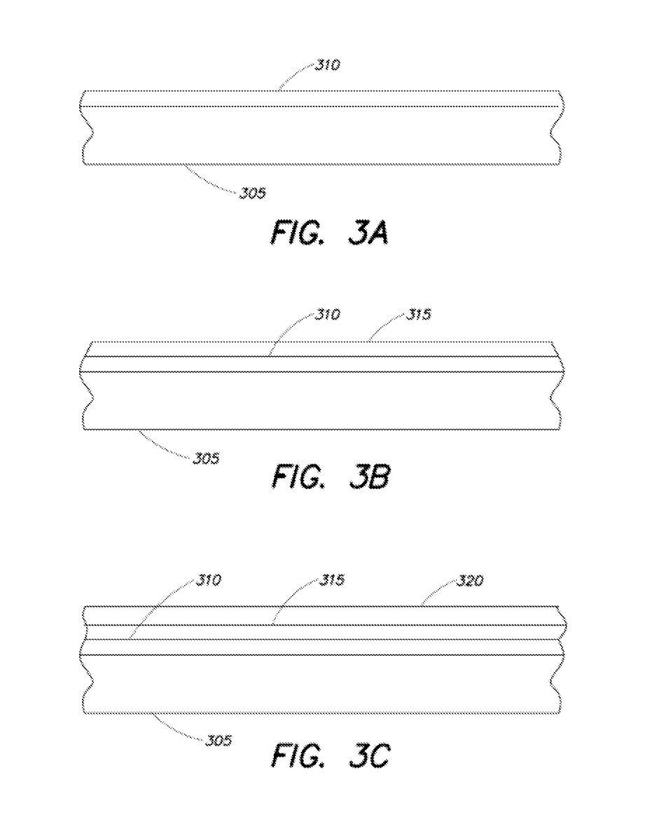

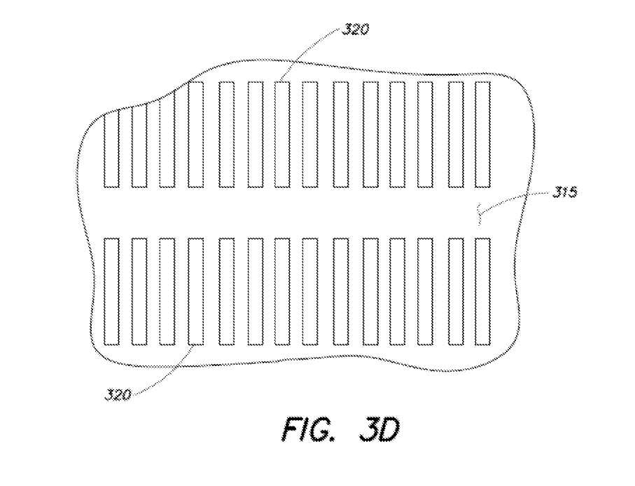

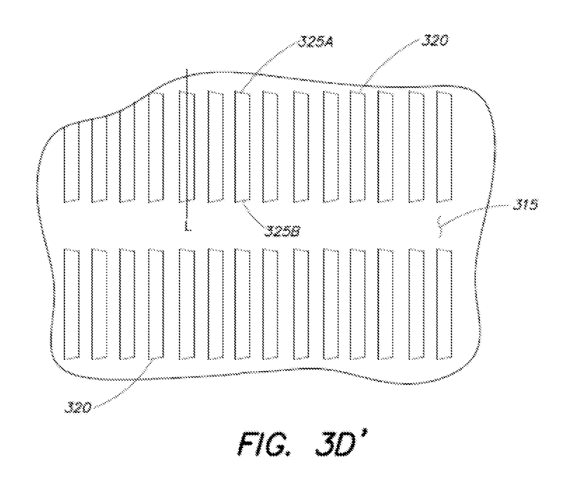

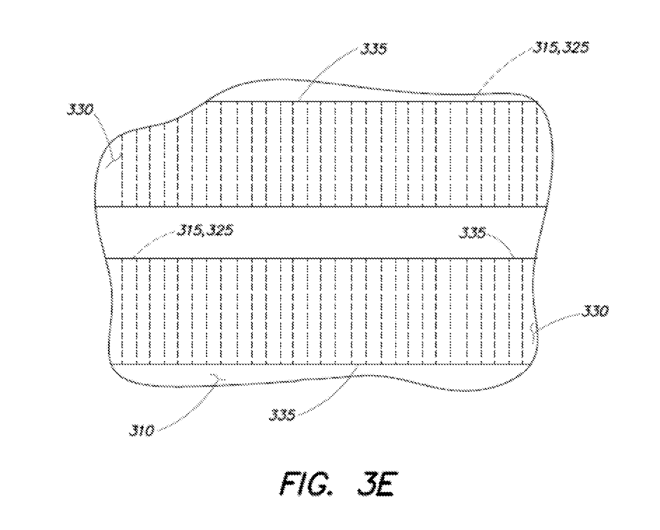

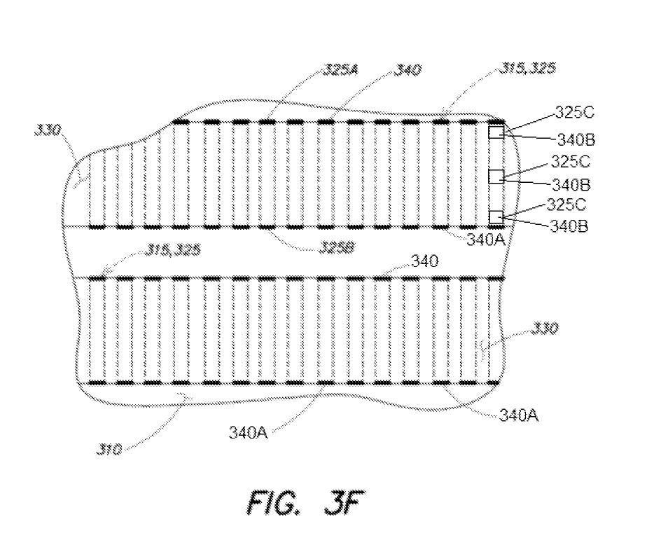

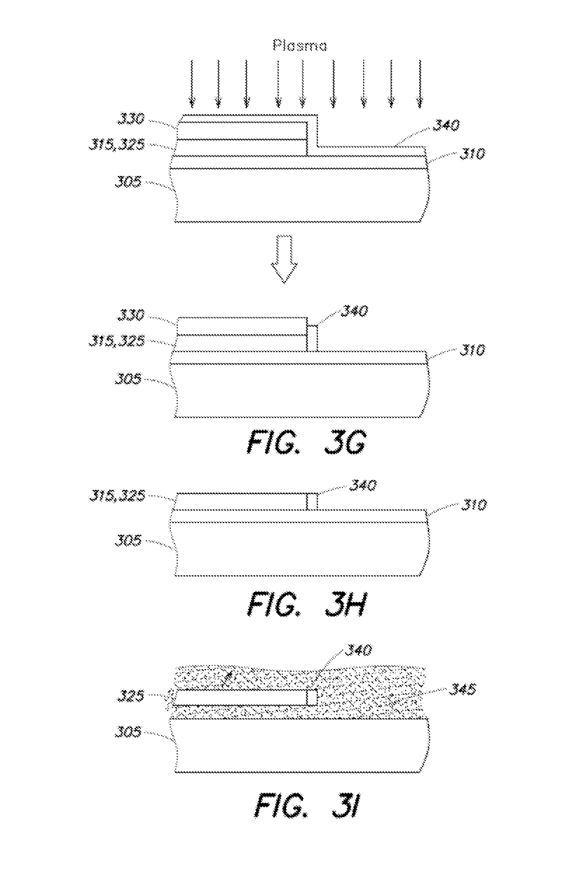

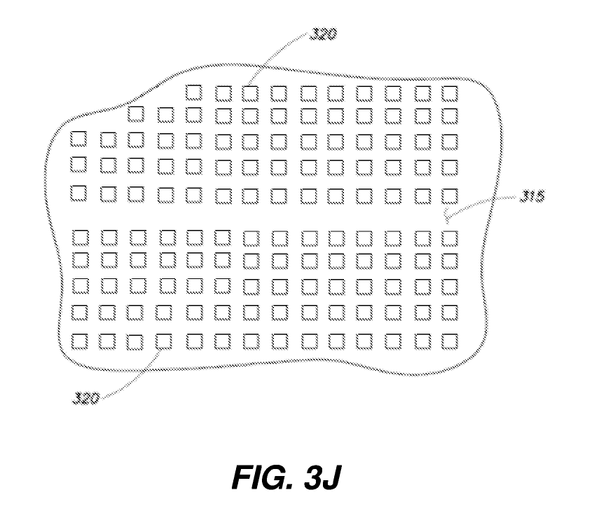

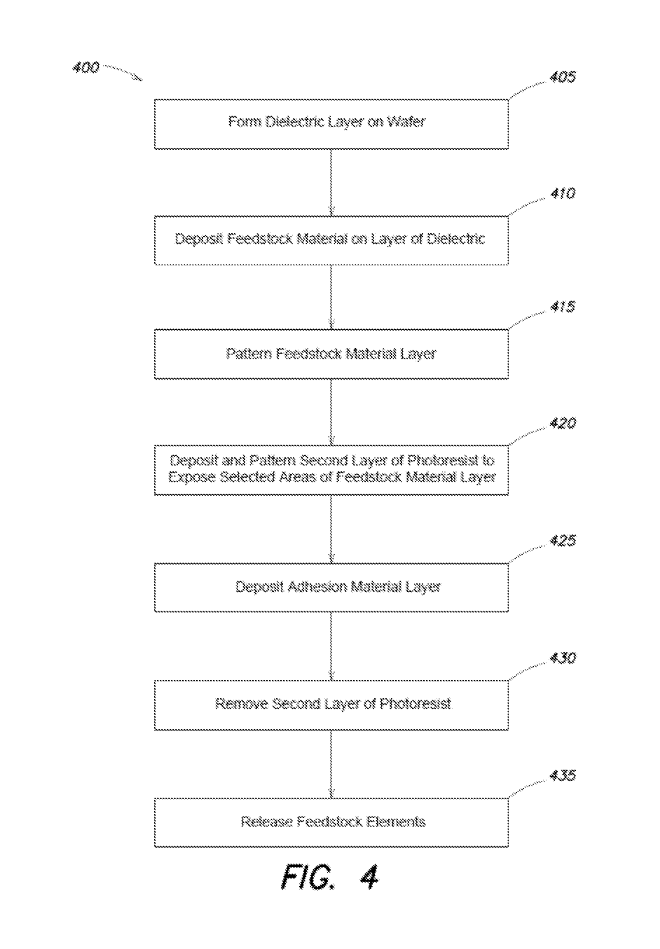

Micro-scale feedstock elements utilized in forming 2-D and 3-D structures disclosed herein may be formed using processes similar to those used in the fabrication of electronic devices in the semiconductor industry and/or micro electro mechanical system (MEMS) devices. One example of a method 400 for forming micro-scale feedstock elements utilized in forming 2-D and 3-D structures disclosed herein is described in the flowchart of FIG. 4 and the schematic diagrams in FIGS. 3A-3J.

In act 405, a substrate, for example, a silicon wafer 305 (or alternatively, sapphire, a glass wafer, a piezoelectric material, quartz or another insulator, or another substrate material desired for a particular implementation) is provided and a sacrificial layer of dielectric 310 for example, silicon dioxide (SiO₂) or silicon nitride (Si₃N₄ (which may be utilized when forming a feedstock element from SiO₂)) is grown on the face of the silicon wafer 305 using a chemical vapor deposition (CVD) or diffusion process in a diffusion furnace as known in the semiconductor fabrication arts (See FIG. 3A, illustrating a portion of the wafer 305 and layer of dielectric 310, not drawn to scale). The layer of dielectric 310 may be between about 100 nm and about 50 μm thick, although this range is an example only and is not intended to be limiting. As discussed below, in some embodiments a sacrificial polymer layer, for example, photoresist or polyvinyl alcohol (PVA) may be used in addition to or in place of the dielectric 310.

In act 410 (FIG. 3B), a layer 315 of the desired feedstock material is then deposited on the layer of dielectric 310. The method of deposition is dependent on the type of feedstock material. For example, if the feedstock material is Si, SiO₂, or Si₃N₄, the feedstock material may be deposited via a CVD process, a spin-on glass process, or grown in a diffusion furnace. If the feedstock material is a metal it may be deposited using an electroplating process or a physical vapor deposition process such as sputtering or evaporative deposition. Photoresists or other polymers may be deposited on the layer of dielectric 310 using a spin-on process, optionally followed by a bake process to remove volatile solvents from the photoresist or other polymer. These and other processes for depositing various materials on a layer of dielectric 310 on a wafer are well known in the semiconductor fabrication arts and will not be described in detail herein. The layer 315 of feedstock material may be between about 0.1 μm and about 100 μm thick, although this range is an example only and is not intended to be limiting.

In act 415, the layer 315 of the feedstock material is patterned. Patterning of the layer 315 of the feedstock material may be accomplished using known methods of patterning of features on a semiconductor wafer. For example, a layer of photoresist 320 may be deposited conformally over the layer 315 of the feedstock material by spin coating and prebaked to drive off excess photoresist solvent. (FIG. 3C.) The layer of photoresist 320 is then exposed to crosslinking radiation (for negative photoresist), for example, ultraviolet light, through a photomask to define patterns in the crosslinked layer of photoresist 320 having dimensions desired for the micro-scale feedstock elements and optionally subjected to a post-exposure bake to help reduce standing wave phenomena caused by the destructive and constructive interference patterns of the crosslinking radiation. The non-crosslinked photoresist is then removed in a developing process by exposure to a developer chemical, for example, a developer such as tetramethylammonium hydroxide, and optionally subjected to a hard bake to solidify the remaining photoresist. The removal of the non-crosslinked photoresist exposes portions of the layer 315 of the feedstock material (FIG. 3D, illustrating an enlarged plan view of a portion of the wafer, aspect ratios of portions of layer 315 covered by remaining photoresist 320 not shown to scale) which is then etched using dry and/or wet etch processes depending on the type of feedstock material to form the micro-scale feedstock elements 325 from the layer 315 with the desired dimensions. The remaining crosslinked photoresist 320 is them removed by chemical resist stripping and/or by thermal decomposition in an ashing process and the wafer 305 may be cleaned, for example, in a sulfuric acid/hydrogen peroxide solution as is known in the semiconductor fabrication arts. In some embodiments, for example, as illustrated in FIG. 3D′ (also shown in feedstock elements L1 in FIG. 1F), one or both ends 325A, 325B of the micro-scale feedstock elements 325 may be patterned at an angle relative to a lengthwise longitudinal axis L of the feedstock elements 325 (for example, between 0 and about 45 degrees) to facilitate attaching the feedstock elements 325 to a substrate or to other feedstock elements at an angle.

In act 420 a second layer of photoresist 330 is then deposited on the micro-scale feedstock elements 325 and patterned such that only portions of the feedstock elements 325 that are desired to be functionalized are exposed. (FIG. 3E.) In some embodiments, after patterning of the second layer of photoresist 330, end portions of the feedstock elements 325 that are exposed are etched away so only end surfaces 335 of the micro-scale feedstock elements 325 are exposed.

In act 425 an adhesion material 340 to which a click chemical group and associated binder molecule is later to be bonded is deposited on the exposed portions of the feedstock elements 325. (FIG. 3F.) In some embodiments, the binder molecule will attach directly to the exposed feedstock while the rest of the feedstock is protected under the photoresist. In some embodiments, material 340 includes or consists of a metal or semiconductor, for example, gold, silicon, silicon dioxide, iron or iron oxides, nickel, or an organic polymer. In some embodiments, the material 340 is conformally deposited by CVD or an evaporation deposition process. In other embodiments, where the exposed portions of the feedstock elements 325 are exposed at their upper surfaces, or if the wafer 305 can be oriented in a deposition chamber of a sputtering tool to expose the exposed portions of the micro-scale feedstock elements 325 in a direction toward a sputtering material target, a sputtering process may be utilized to deposit the material 340. The second layer of photoresist 330 is then removed, for example, by wet chemical etching which will also remove the material sputtered onto the photoresist, leaving the ends of the feedstock elements coated in the sputtered material. In some embodiments, act 425 is repeated to deposit different materials 340, 340A on different portions of the micro-scale feedstock elements 325, for example, different materials at different ends 325A, 325B of the feedstock elements 325, although the same material(s) may be deposited on each of the different ends 325A, 325B of the feedstock elements 325. Further, as illustrated in FIG. 3F portions 325C of upper surfaces and/or side surfaces of the micro-scale feedstock elements 325 may be exposed by lithographic photoresist patterning and a similar or a different material 340B that may bond to a different click chemical moiety than materials 340, 340A may be deposited on the portions 325C of upper surfaces and/or side surfaces of the micro-scale feedstock elements 325. In some embodiments, as illustrated in FIG. 3F, the material 340 (and optionally, 340A) selectively deposits on exposed portions of the feedstock elements 325. In alternate embodiments, a masking material is used instead of adhesion material 340 to define areas of the micro-scale feedstock elements 325 to which a click chemical group and associated binder molecule is later to be prevented from bonding to.

In other embodiments, the material 340 deposits conformally over the second layer of photoresist 330, the exposed portions of the feedstock elements 325, and the exposed surface of dielectric layer 310, in which instance a further photoresist layer may be deposited to cover the portions of the feedstock elements 325 onto which the material 340 was deposited and expose the surface of dielectric layer 310 on which the material 340 was deposited so that the material 340 may be etched off of the surface of dielectric layer 310 on which the material 340 was deposited, for example, with a wet etch. The further layer of photoresist would then be removed. Alternatively or additionally, material 340 deposited on the exposed surface of dielectric layer 310 may be removed with an anisotropic dry etch (for example, an argon plasma etch) with or without providing a layer of photoresist to protect the ends of the feedstock elements 325 onto which the material 340 was deposited. (See FIG. 3G, a schematic cross sectional illustration through a portion of one of the feedstock elements and adjacent structures.)

In act 430, the second layer of photoresist 330 is removed, for example, by thermal decomposition and/or chemical dissolution. Portions of the material 340 adhered to the second layer of photoresist 330 may also be removed in this act, resulting in the feedstock element layer 315 including the material 340 attached to the feedstock elements remaining on the layer of dielectric 310. (FIG. 3H.)

In act 435, the micro-scale feedstock elements 325 are released from the wafer 305 by dissolving or etching away the dielectric layer 310 by exposure to a wet etching agent 345, for example, hydrofluoric acid if the dielectric layer 310 is SiO₂, phosphoric acid if the dielectric layer 310 is Si₃N₄, or other suitable etching agents selected depending on the material of the dielectric layer 310. In act 435 the released micro-scale feedstock elements 325 are collected, for example, by filtering the etching agent 345 used to release them and optionally washed to neutralize the etching agent.

Various modifications may be made to the above process. For example, instead of a layer of dielectric 310 being deposited on the silicon wafer 305 and then removed by chemical etching, a layer of a polymer, for example, a photoresist, polyimide, or another polymer, may be deposited on the silicon wafer 305 and later removed by, for example, exposure to a solvent (ethylene glycol, gamma-butyrolactone, cyclopentanone, N-Methyl-2-pyrrolidone, or other known solvents) and/or by thermal decomposition as is known in the semiconductor fabrication arts to release the formed micro-scale feedstock elements. Alternatively, polyvinylalcohol (PVA), which is soluble in water, could be used as layer 310 and subsequently removed by exposure to water in act 435. The photoresist 320 may be positive photoresist that becomes soluble when exposed to radiation through the photomask and thus is exposed in areas other than those having the desired shapes for the micro-scale feedstock elements 325. In some embodiments, the layer 315 from which the feedstock elements 325 are formed may itself be a photoimagable polymer, for example, SU-8, in which instance the first photoresist layer 220 may not be necessary and the layer 315 may be directly patterned by exposure to patterning radiation and development in developer solution. In some embodiments, differently sized and/or shaped micro-scale feedstock elements may be formed concurrently on the same wafer while in other embodiments only micro-scale feedstock elements having same dimensions are formed on a single wafer.

In further embodiments, for example, instead of forming rod or bar shaped micro-scale feedstock elements 325, the feedstock elements 325 may be formed as block or cube shaped elements. For example, comparing FIG. 3J to FIG. 3D, the photoresist 320 illustrated in FIG. 3J may be lithographically patterned substantially as squares instead of as rods or bars as illustrated in FIG. 3D. The layer 315 could then be etched to form block or cube shaped feedstock elements 325. The block or cube shaped feedstock elements 325 may be processed similarly to the rod or bar shaped micro-scale feedstock elements 325 as described above to deposit different materials on different faces of the block or cube shaped feedstock elements 325 to which different click chemical groups may be bound. The block or cube shaped feedstock elements 325 may be formed of various desired materials, for example, one or more of silicon, silicon dioxide, silicon nitride, silicon carbide, alumina, titania, zinc oxide, tungsten, SU-8 photoresist or other organic or inorganic polymers, biologically-based materials, for example, chitosan, or other materials selected based on, for example, desired mechanical, thermal, optical, electrical, magnetic, and/or chemical properties, similar to the rod or bar shaped micro-scale feedstock elements 325. It should be appreciated that the feedstock elements 325 may alternatively be formed as three dimensional regular or irregular prisms other than blocks or cubes, for example, rectangular prisms, triangular prisms, pentagonal prisms, hexagonal prisms, etc. with different sides having the same or different materials deposited on them to which the same or different click chemical groups may bond.

In some embodiments, block or cube shaped feedstock elements, or elements having different three dimensional shapes, for example, spheres or regular or irregular prisms other than blocks or cubes may be utilized as linker elements 1135, discussed below, that may be used as an intermediate element to join other feedstock elements (or a feedstock element to a substrate.) The linker elements 1135 may be formed with shapes, for example, with walls disposed at desired angles relative to one another that provide for feedstock elements joined with the linker elements 1135 to be joined at pre-determined desired angles. The linker elements 1135 may be formed with sides, or functionalized portions thereof, having sizes that allow for a predetermined number of feedstock elements, for example, between 1 and about 10 or more to bond to the sides of the linker elements 1135. The predetermined number of feedstock elements may be defined by a size of the bonding areas of the predetermined number of feedstock elements relative to the size of the sides, or functionalized portions thereof, of the linker elements 1135.

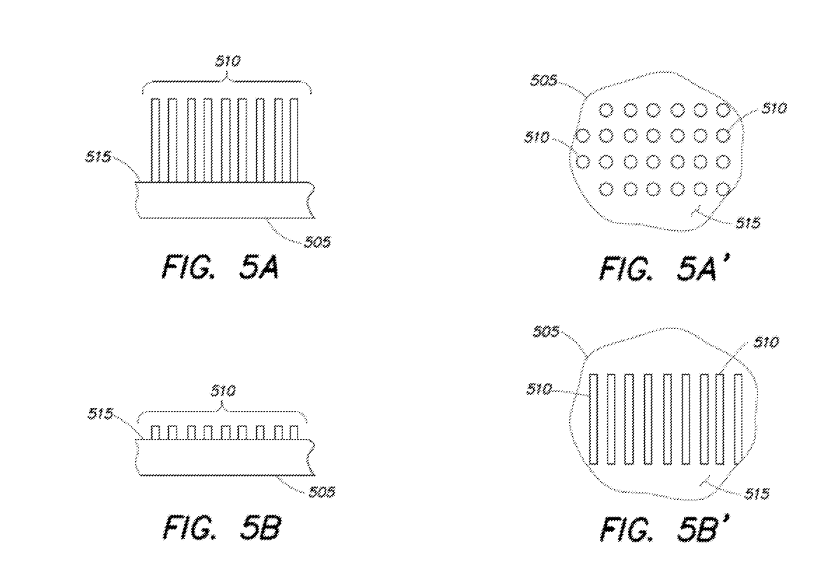

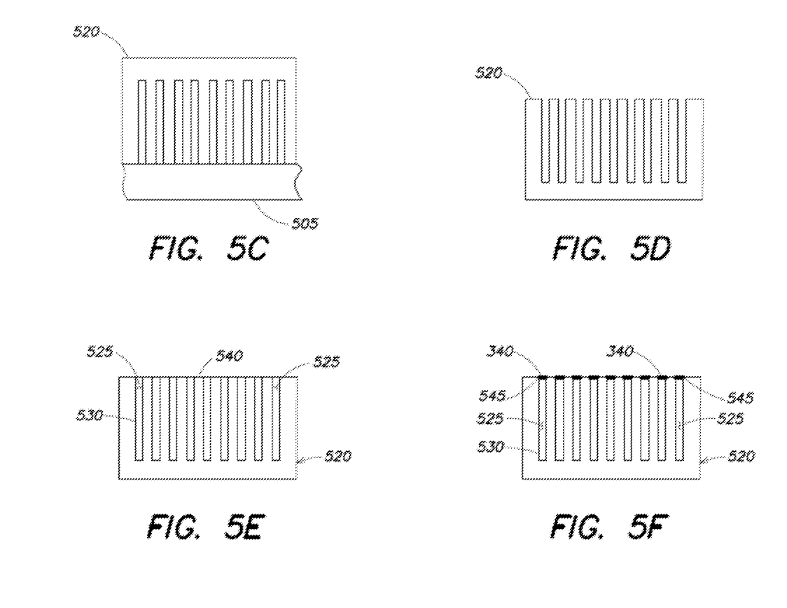

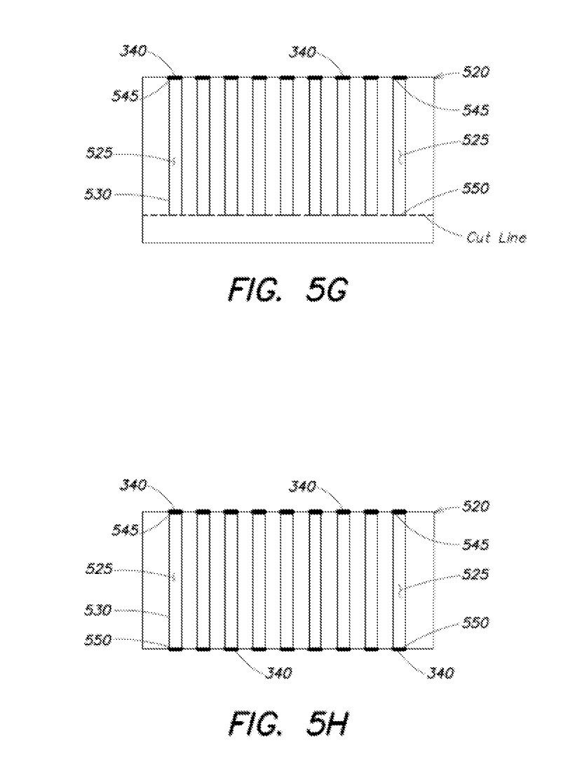

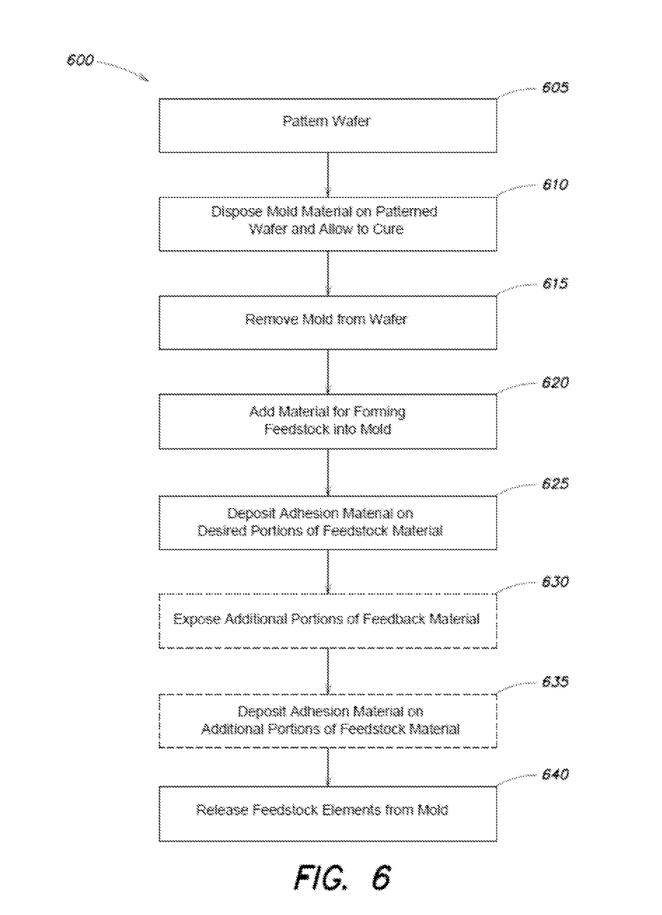

Another embodiment of a process 600 for forming micro-scale feedstock elements 325 is described with reference to FIGS. 5A-5H and the flowchart of FIG. 6. In act 605 a material, for example a semiconductor wafer 505 is patterned to exhibit an array of structures 510 having dimensions substantially similar to a desired micro-scale feedstock element 325 to be formed. In some embodiments, as illustrated in FIGS. 5A and 5A′, the structures may be oriented perpendicular to the surface 515 of the semiconductor wafer 505. In other embodiments, as illustrated in FIGS. 5B and 5B′, the structures may be oriented parallel to and disposed on the surface 515 of the semiconductor wafer 505. The structures 510 may be substantially cylindrical, substantially rectangular in cross-section or any other shape and with dimensions desired for the micro-scale feedstock elements 325. For example, although illustrated as high aspect ratio cylinders or rods in FIGS. 5A, 5B, 5A′, and 5B′, structures 510 may have low aspect ratios and be in the form of, for example, pucks, blocks, cubes, or other regular or irregular prisms.

In act 610, a mold material, for example, wax, silicone, an epoxy-based material, or another mold material known in the art is deposited on the array of structures and allowed to cure to form a mold 520. (FIG. 5C.) In some embodiments, a release agent is deposited on the array of structures prior to deposition of the mold material. Examples of release agents include, for example, vapor-deposited polytetrafluoroethylene, or vapor-deposited dimethyldichlorosilane available as PlusOne™ Repel-Silane ES from GE Healthcare Life Sciences.

In act 615, the cured mold 520 is removed from the semiconductor wafer 505 and array of structures 510. (FIG. 5D.)

In act 620 a desired material 525, in a liquid or slurry form, is deposited in the impressions 530 in the mold 520 formed by the array of structures 510 and excess material 525, for example, from the surface 540 of the mold is removed. (FIG. 5E.) The material 525 is allowed to solidify or cure. Heat and/or radiation, for example, UV light, actinic radiation, or other forms of radiation, may be applied to the material 525 to facilitate and/or accelerate solidification or curing.

In act 625 a layer of adhesion material 340, for example, any one or more of the adhesion materials 340 discussed above is deposited on desired portions of the solidified material 525, for example, on end portions 545 exposed in the impressions 530 in the mold 520. (FIG. 5F.) In some embodiments, the one or more of the adhesion materials 340 are deposited by a physical deposition method, for example sputtering or evaporative deposition. In other embodiments, the one or more of the adhesion materials 340 are deposited by a screen printing or other deposition method.

In some embodiments where it is desired to deposit the one or more of the adhesion materials 340 on additional portions of the solidified material 525, the mold 520 may be cut to expose the additional portions, for example, other end portions 550 of the solidified material 525. (FIG. 5G, optional act 630.) The one or more of the adhesion materials 340 may then be deposited on the additional portions using a similar method as the or more of the adhesion materials 340 was deposited on the first desired portions. (FIG. 5H, optional act 635.) In some embodiments, different types of adhesion materials 340, for example, materials that bond to different and/or complimentary click chemical groups may be deposited on the different portions of the solidified material 525.

In act 640, the solidified material 525 with the deposited adhesion material(s) 340 is removed from the mold 520, for example by melting of the mold material, dissolution of the material of the mold in a solvent, by cutting the solidified material 525 from the mold, or by other methods known in the art, resulting in a plurality of free micro-scale feedstock elements 325 which are then collected for later use.

In some embodiments disclosed herein, structures are formed including nanotubes as micro-scale feedstock elements. The nanotubes may be utilized for any of the feedstock elements disclosed herein. The nanotubes may comprise or consist of carbon, boron, or other elements. Carbon nanotubes may have diameters as small as a few nanometers. Carbon nanotubes may be formed by a CVD process in which the carbon nanotubes form on metal catalyst particles, for example, particles of nickel, cobalt, iron, or a combination thereof. The catalyst particles can stay at the tips of the growing nanotube during growth, or remain at the nanotube base during growth. The catalyst particles are often removed from carbon nanotubes available from various suppliers. However, in some embodiments the catalyst particles may be retained on the carbon nanotubes and used as the adhesion material 340 to which click chemicals and associated binder molecules may be adhered to facilitate attachment of the carbon nanotubes to other micro-scale feedstock elements. In other embodiments, defects, for example, carboxylic acid may be preferentially generated on end portions of carbon nanotubes to facilitate bonding to an adhesion material, for example, an amide group bonded to a substrate or to another feedstock element.

Nano-Scale Feedstock Fabricated with Methods Utilizing Block Copolymers

In some embodiments, it may be desirable to form feedstock elements for the fabrication of micro or macro-scale structures having dimensions smaller than those achievable with conventional lithographic processes, for example, rods, bars, cylinders, or prisms with widths or diameters of about 20 nm or less. It may be desirable to form such nanoscale feedstock elements from materials such as silicon, silicon dioxide, titanium dioxide, zinc oxide, tungsten, or other materials having desired physical, chemical, optical, electrical, or magnetic properties. In some embodiments, one may capitalize on the ability of various types of di-block or multi-block copolymers to self-assemble into nanoscale structures in methods to form such nanoscale feedstock elements.

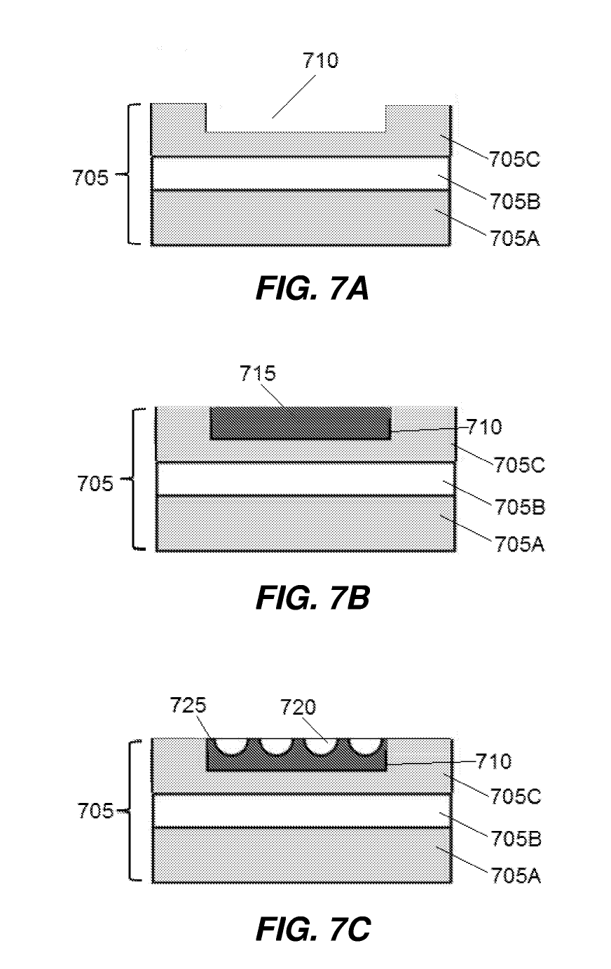

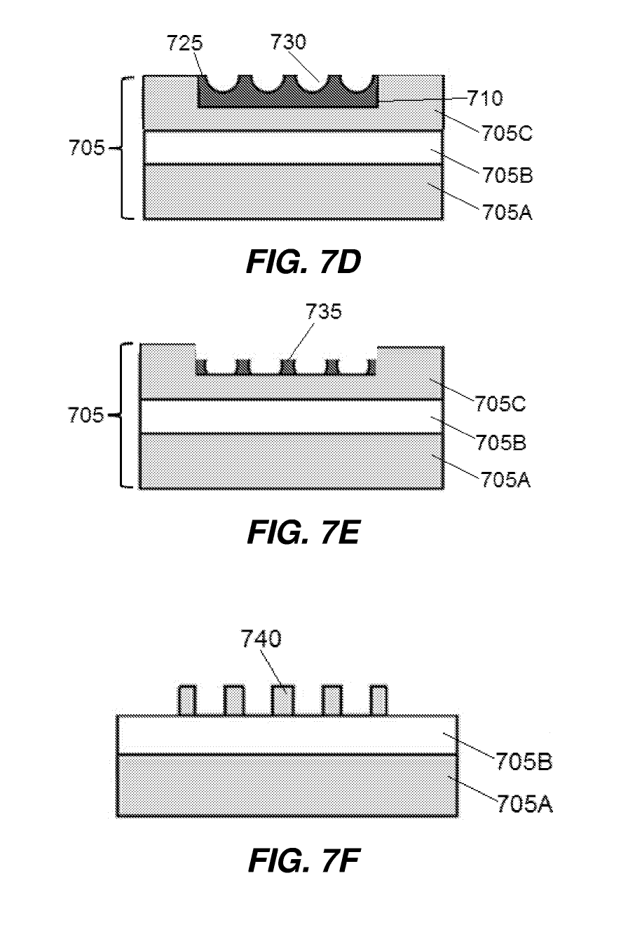

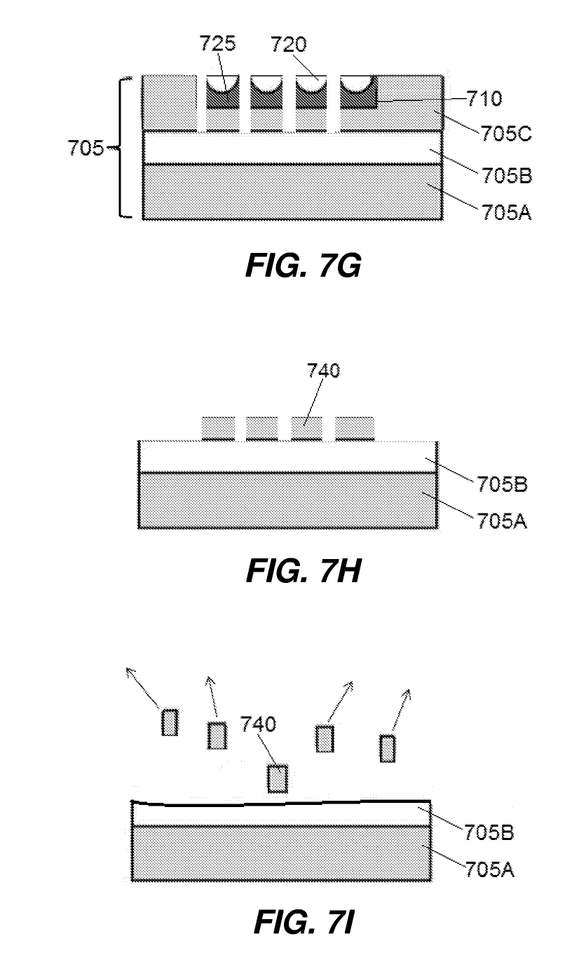

In one specific example of block copolymer self assembly, a diblock copolymer composed of 70% polystyrene (PS) and 30% poly(methyl-methacrylate (PMMA) (poly(styrene-b-methyl methacrylate), referred to hereinafter as 70:30 PS-b-PMMA) with a total molecular weight of 64 kg/mol is deposited into a trench having a width of from about 100 nm to about 600 nm etched into the surface of a substrate, for example, silicon or silicon dioxide. The 70:30 PS-b-PMMA will spontaneously form hexagonal lattices of 20 nm diameter cylindrical PMMA domains in a matrix of PS. The PMMA may be removed from the cylindrical domains by immersion in acetic acid, leaving behind a pattern of 20 nm wide trenches from which nanoscale feedstock elements may be formed. An example of a nanoscale feedstock assembly method utilizing 70:30 PS-b-PMMA is illustrated in FIGS. 7A-7I. In FIG. 7A a lithographically defined trench 710 is formed in an upper surface of a substrate 705. In the illustrated embodiment, the substrate 705 is a multilayer substrate including a lower layer 705A of, for example, silicon, an intermediate layer 705B of silicon oxide or silicon nitride, and an upper layer 705C of, for example, silicon or silicon oxide. In embodiments in which the upper layer 705C may be selectively etched relative to the lower layer 705A, the intermediate layer 705B may be omitted. The upper layer 705C may be, for example, about 20 nm thick, or a thickness corresponding to the diameter of the diblock copolymer domain that will serve as an etch mask as described below. The trench 710 may have a depth of, for example, about 40 nm or approximately the natural period of the diblock copolymer pattern, a width of, for example, about 500 nm, and a length that can be from several microns to the width of a chip or a semiconductor wafer (up to several hundred mm) on which the trench is formed. It should be appreciated that instead of etching a trench, an area having similar dimensions as the trench could be defined by depositing and patterning walls of photoresist or another material on the upper surface of the substrate 705.

In FIG. 7B, a liquid phase 70:30 PS-b-PMMA copolymer 715 is deposited on the top of the substrate 705, for example, using spin deposition, and fills the trench 705. The deposited 70:30 PS-b-PMMA copolymer 715 is then annealed, for example, at about 250° C. for about 8 h and then cooled to room temperature to obtain self assembled PMMA patterns in the annealed copolymer. The walls 710 of the trench 705 initiate the assembly of polymer domains in the 70:30 PS-b-PMMA copolymer resulting in the formation of PMMA half cylinders 720 in a PS matrix 725 in the trench 705, as shown in FIG. 7C. The PMMA half cylinders are then removed by immersion of the substrate 705 in acetic acid, leaving behind recesses 730 (FIG. 7D). A brief O₂ plasma etch removes the PS below the recesses 730 exposing the surface of layer 705C between islands 735 of remaining PS. (FIG. 7E). A second plasma etch using, for example, a SF₆+O₂ plasma removes all of layer 705C that was not masked by the PS islands 735, resulting in the formation of nanorods 740 of the material of layer 705C, for example, silicon nanorods. The nanorods 740 may then be covered by patterned photoresist in a similar manner as the feedstock elements 325 as described above with reference to, for example, FIG. 3E and may have click chemical bonding material deposited on their ends and/or portions of their sides as described above with reference to FIGS. 3F-3H.

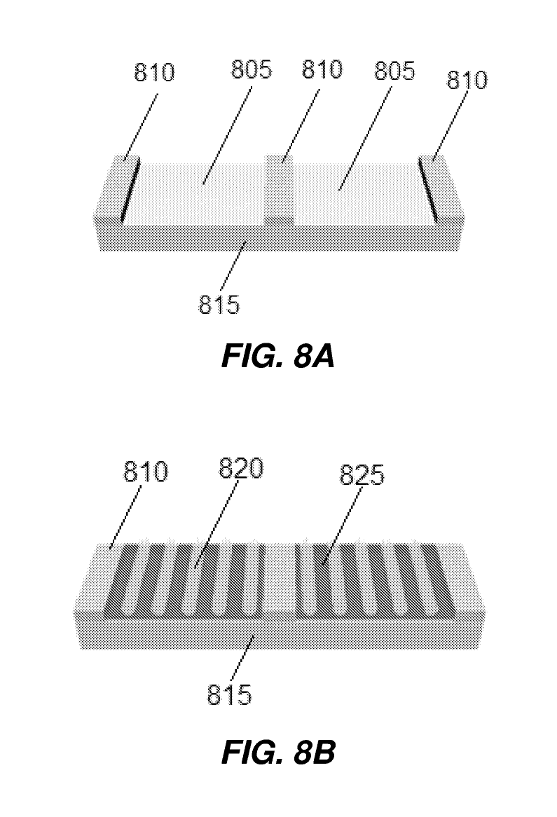

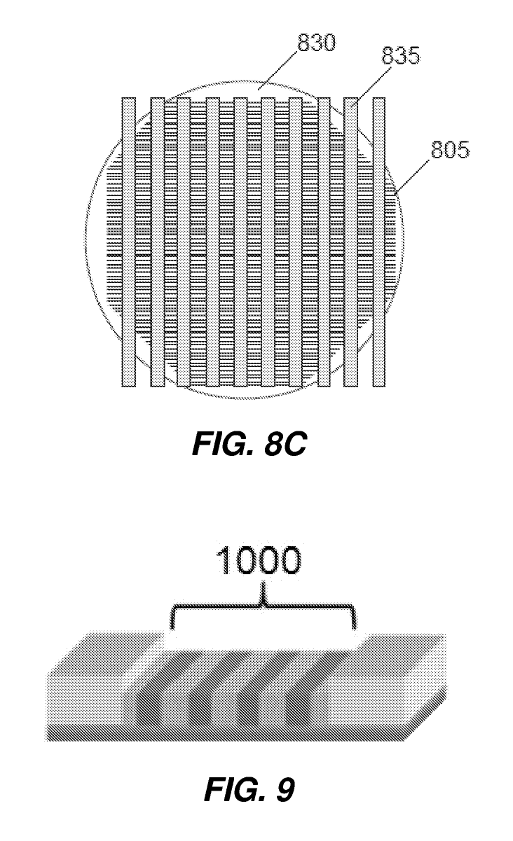

Alternatively, starting at 7C, the PMMA cylinders can be converted to an inorganic material, for example, alumina, through sequential infiltration synthesis. The alumina rods can serve as an etch mask for the PS matrix to expose the layer into which nanorods are to be patterned 705C and as an etch mask for forming the nanorods 740 in the nanorod layer 705C. (See FIG. 7G.) Once the nanorods 740 are defined by etching, the PS matrix and converted PMMA cylinders may be removed with, for example, thermal ashing and/or with a solvent and excess portions of layer 705C may be removed by appropriate lithographic patterning and etching. (See FIG. 7H.) Once nanorods 740 are formed, the layer of nanorods 740 may be lithographically patterned in the direction orthogonal to the lengthwise axis of the nanorods 740 and then etched as described below and shown in FIG. 8C to set the length of the nanorods. These nanorods 740 would be embedded in a photoresist matrix after their lengths are set with only their ends exposed and could be selectively functionalized only on the ends before release.

The nanorods 740 may then be released from the remainder of the substrate 705 by removing any remaining photoresist using known photoresist stripping methods, and by dissolving or etching away layer 705B by exposure to a wet etching agent, for example, hydrofluoric acid if the layer 705B is SiO₂, phosphoric acid if the layer 705B is Si₃N₄, or other suitable etching agents selected depending on the material of the layer 705B. If the underling substrate 705A is silicon and there is no intermediate layer, a dry XeF₂ etch or a wet KOH or TMAH etch could release the nanorods 740. The released nanorods 740 may be collected, for example, by filtering the etching agent used to release them and optionally washed to neutralize the etching agent.

Various modifications may be made to the above process. For example, instead of layer 705B being silicon oxide or silicon nitride, layer 705B may be a layer of a polymer, for example, a photoresist, polyimide, or another polymer, that may be deposited on the layer 705A and later removed by, for example, exposure to a solvent (ethylene glycol, gamma-butyrolactone, cyclopentanone, N-Methyl-2-pyrrolidone, or other known solvents) and/or by thermal decomposition as is known in the semiconductor fabrication arts to release the formed nanorods 740. Alternatively, polyvinylalcohol (PVA), which is soluble in water, could be used as layer 705B and subsequently removed by exposure to water to release the formed nanorods 740. Further, the diameters of the PMMA domains and thus, the nanorods 740 formed in this process may be adjusted by adjusting factors such as the amount of PS v. PMMA in the PS-b-PMMA copolymer. A larger percentage of PMMA may result in larger diameter PMMA domains and a lesser percentage of PMMA may result in smaller diameter PMMA domains than in the example described above. In some embodiments, the diameter of the PMMA domains may vary from about 5 nm to about 50 nm.

The shape of the PMMA domains may be adjusted by adjusting the depth of the trench 710. As described above, a trench with a depth of about 25 nm would result in half cylinder domains of PMMA being formed using 70:30 PS-b-PMMA copolymer. Increasing the depth of the trench and/or decreasing the percentage of PMMA in the PS-b-PMMA copolymer may provide for full cylinders of PMMA domains fully embedded in a PS matrix to be formed.

Another method of forming nanoscale feedstock elements, for example, nanorods of materials including, but not limited to one or more of alumina, titania, zinc oxide, or tungsten utilizing block copolymers involves Sequential Infiltration Synthesis (SIS) of self assembled domains in a block copolymer. An example of this method is illustrated in FIGS. 8A-8C.

FIG. 8A illustrates a portion of a substrate, for example, a silicon substrate upon which nanoscale feedstock elements are to be formed. Film receiving regions 805 are defined by patterned lines of photoresist 810 on an upper surface of the substrate 815. A liquid block copolymer, for example, PS-b-PMMA copolymer is deposited on the substrate 815, for example, by spin deposition, and fills at least a portion of the film receiving regions 805. The block copolymer is annealed and forms separate polymer domains, for example, PMMA rods 820 in a matrix 825 of PS, as illustrated in FIG. 8B. The PMMA rods may have diameters of from about 10 nm to about 20 nm, although these dimensions should not be considered limiting.

The film receiving regions 805 may extend entirely or substantially entirely across the width of a wafer 830 formed of the substrate 815 (FIG. 8C). The length of nanoscale feedstock elements to be formed from the annealed block copolymer may be defined by a patterning a photoresist 835 on the wafer, removing exposed areas of the annealed block copolymer by etching, for example, with an O₂ plasma etch, and then removing the photoresist 835. This etch could alternatively be performed after the SIS steps below to define the length of the nanorods 740 formed by this method.

Nanoscale feedstock elements comprising, consisting, or consisting essentially of one of, for example, alumina, titania, zinc oxide, or tungsten may then be formed from the PMMA rods 820 of the annealed block copolymer by SIS. In the SIS process, the annealed block copolymer is first exposed to a vapor including a metal precursor, for example, one of TiCl₄, AlCl₃, Al(CH₃)₃, or another known metal precursor that diffuses through the PS matrix and reacts with carbonyl groups in the PMMA domains. Non-coordinated excess metal precursors may be removed from annealed block copolymer, for example, by a purge step with high purity N₂. The first monolayer of metal precursors that are bound to the carbonyl groups of the PMMA provide reactive sites to which materials introduced in vapor form to the annealed block copolymer, for example, in an Atomic layer Deposition (ALD) process, bond. For example, to form Al₂O₃ nanorods, the annealed PS-b-PMMA copolymer is exposed to Al(CH₃)₃/H₂O vapor in an ALD deposition apparatus. A coordination reaction occurs between the Al(CH₃)₃ and carbonyl groups in the PMMA, which is followed by H₂O hydrolysis and results in the formation of a first monolayer of an Al—OH species in the PMMA domain. Subsequent ALD cycles of Al(CH₃)₃/H₂O exposure (for example, with 300 s of N₂ purge and 60 s of Al(CH₃)₃/H₂O exposure per cycle) result in the formation and bonding of Al₂O₃ to the first monolayer of Al—OH species and building up of an Al₂O₃ cylinder layer by layer in the PMMA domain. A number of ALD cycles may be performed to grow the Al₂O₃ cylinder to a desired diameter. For example, after 10 ALD cycles, an Al₂O₃ cylinder having a diameter of about 30 nm will have been formed.

After the Al₂O₃ cylinders have been grown to a desired diameter, the Al₂O₃ cylinders may be functionalized with materials to which click chemical groups will bond or functionalized directly with desired click chemical groups. In one embodiment, end faces of the PS matrix including the formed Al₂O₃ cylinders may be etched, for example, utilizing an O₂ plasma etch to expose ends of the Al₂O₃ cylinders. A desired material may be deposited on the ends of the Al₂O₃ cylinders by, for example sputtering or CVD. Different materials may be deposited on different ends of the Al₂O₃ cylinders so that different or complimentary click chemical groups may be bonded to the opposite ends of the Al₂O₃ cylinders.

The PS matrix is then removed to release the Al₂O₃ nanoscale feedstock elements, for example, by dissolution of the PS in an appropriate solvent. The released Al₂O₃ nanoscale feedstock elements are then collected, for example by filtering the solvent used to dissolve the PS.

A similar method is used to form TiO₂ cylinder nanoscale feedstock elements from annealed PS-b-PMMA copolymer. To form the TiO₂ cylinder nanoscale feedstock elements, the annealed PS-b-PMMA copolymer may be exposed to a TiCl₄/H₂O vapor in an ALD deposition apparatus. The TiCl₄ coordinates to carbonyl groups in the PMMA domains of the annealed PS-b-PMMA copolymer and hydrolyze to form Ti—OH. The Ti—OH species serve as reactive sites for subsequent TiCl₄/H₂O ALD deposition cycles to build up the TiO₂ cylinder nanoscale feedstock elements.

SiO₂ cylinder nanoscale feedstock elements may be formed from annealed PS-b-PMMA copolymer by Al-catalyzed SiO₂ ALD. The annealed PS-b-PMMA copolymer is first exposed to Al(CH₃)₃/H₂O vapor in an ALD deposition apparatus. A coordination reaction occurs between the Al(CH₃)₃ and carbonyl groups in the PMMA, which is followed by H₂O hydrolysis and results in the formation of Al—OH species in the PMMA domain. Subsequent ALD cycles using silanol vapor and N₂ purges (for example, alternating silanol exposures for 400 s and N₂ purges for 1,200 s) will result in the silanol reacting with the Al—OH species to form SiO₂.

ZnO cylinder nanoscale feedstock elements may be formed from annealed PS-b-PMMA copolymer by Al-catalyzed ZnO ALD which involves first forming Al—OH species in the PMMA domains of the annealed PS-b-PMMA copolymer by Al(CH₃)₃/H₂O ALD and subsequent diethyl zinc ALD cycles (for example, alternating diethyl zinc exposures for 300 s and N₂ purges for 300 s). Tunsten cylinder nanoscale feedstock elements may be formed from annealed PS-b-PMMA copolymer by Al-catalyzed W ALD which involves first forming Al—OH species in the PMMA domains of the annealed PS-b-PMMA copolymer by Al(CH₃)₃/H₂O ALD and subsequent alternating ALD exposures to Si₂H₆ and WF₆ species.

It should be appreciated that various modifications may be made to the SIS process described above. PS-b-PMMA block copolymer is only one example of a block copolymer which self assembles into different polymer domains which may be utilized to form nanoscale feedstock elements. Various other known block copolymers that may be suitable for forming nanoscale feedstock elements via a SIS process may include, for example, poly(3-hexylthiophene)-b-perylene diimide acrylate, poly(3-hexylthiophene)-b-vinyl pyridine, poly(3-hexylthiophene)-b-lactide, polystyrene-b-polyethyleneoxide, poly(styrene-b-isoprene), poly(styrene-b-butadiene-b-methyl methacrylate), poly(styrene-b-(ethylene-co-butylene)-b-methylmethacrylate), poly(styrene-b-4-vinylpyridine), poly(isoprene-b-ethylene oxide), poly(styrene-b-ethylene oxide), poly(styrene-b-2-vinylpyridine), poly(styrene-b-hydroxystyrene), poly(styrene-b-n-butyl methacrylate), poly(styrene-b-ferrocenyldimethylsilane), poly(styrene-b-dimethylsioxane), poly[styrene-b-(ethylene-alt-propylene)], poly(styrene-b-ethylene butylene-b-styrene), and poly(styrene-b-butadiene). Some of these block copolymers may self-assemble into rectangular shaped bar-like domains 1000 (See FIG. 9) rather than cylindrical rod-shaped domains, providing for bar shaped nanoscale feedstock elements to be formed.

Instead of or in addition to reacting metal precursors to carbonyl groups in PMMA domains of a PS-b-PMMA block copolymer, other metal precursors may be utilized to bond with different polymer units in block copolymer domains of different block copolymers through, for example, metal-ligand coordination, covalent bonding, or other interactions. For example, the pyridine groups in polyvinylpyridine, a common block in many block copolymers, could selectively bind metal compounds including, for example, Al(CH₃)₃, AlCl₃, ZnCl₂, or CdCl₂. The hydroxyl groups in polyacrylic acid, another common block in many block copolymers, could react with Al(CH₃)₃, TiCl₄, or Zn(C₂H₅)₂ to form covalent bonds. Any of these metal precursors may be utilized as precursors for the growth of various materials in a block domain of a block copolymer using, for example, an ALD process as described above.

In any of the methods of forming feedstock elements disclosed above, functionalized portions of the feedstock elements may include one or more long-chain molecules (for example, polyethylene glycol or a polymer or peptide chain) bonded to the feedstock elements between the bodies of the feedstock elements and the click chemical molecule or molecules or click chemical group or groups connected to the feedstock elements. The long-chain molecules may provide flexibility to the click chemical molecules and/or the ability of the different ends of the click chemical molecules to orient in different directions to provide for bonding of other elements to the feedstock elements in different orientations.

### “Click” Chemistry

“Click chemistry” is a term for a type of chemical synthesis used for generating substances quickly and reliably by joining small units together. Click chemistry describes a way of generating products that follows examples in nature, which also generates substances by joining small modular units. The term was coined by K. Barry Sharpless in 1998, and was first fully described by Sharpless, Hartmuth Kolb, and M. G. Finn of The Scripps Research Institute in 2001.

In some embodiments, “click chemistry” reactions are used to join micro-scale or nano-scale feedstock elements to substrates and/or other micro-scale or nano-scale feedstock elements to form embodiments of structures disclosed herein. Feedstock faces or side portions to be joined (and/or feedstock faces and areas of a substrate to be joined) are patterned with complementary chemical groups, referred to herein as A-A′ pairs, B—B′ pairs, C—C′ pairs etc. (where A will bond to A′ but not B, B′, C, or C′, B will bond to B′ but not A, A′, C, or C′, etc.) that will bond them together with covalent, permanent click reactions. Such covalent bonds are stable to variations in solution conditions, temperature, and removal of water, making them a highly robust approach to hierarchical structure assembly.

Various different “click” reactions may be utilized in embodiments of assembly methods and structures disclosed herein. In one example, alkyne (or cyclooctyne) and azide functional groups represent one such A-A′ pair, displaying one of the most efficient, selective and versatile click reactions known, Huisgen 1,3-dipolar cycloaddition. In another example, the Michael addition of thiols to alkenes (i.e. maleimides) may be used as an alternate A-A′ pair. The reaction of aldehydes with alkoxyamines to form oximes provides a third A-A′ pair that is orthogonally reactive. The oxidative coupling of substituted phenols to anisidine derivatives may be used to provide a fourth A-A′ coupling. Biotin and streptavidin functional groups represent another such A-A′ pair. Multiple other A-A′ click chemical pairs are known in the art.

The high reactivity of the click-active functional moieties is incompatible with most traditional lithographic patterning schemes. To overcome this limitation, some embodiments involve conventional microfabrication techniques to bond an intermediate material to portions of a substrate or a micro-scale or nano-scale feedstock element that is used to bond a click chemical group and/or a linker molecule and click chemical group to the substrate or a micro-scale or nano-scale feedstock element. In some embodiments, a surface of a substrate is patterned with a material to which precursors that will bind to the click chemistries will selectively functionalize (e.g. gold surfaces to which thiols will bind, silicon surfaces to which silanes will bind, or iron oxides and other metals to which carboxyl groups will bind). If micro-scale or nano-scale feedstock elements are fabricated in a template or mold as discussed above (for example, as electroplated pillars in a mold or SIS formed feedstock elements in a polymer matrix) functionalization could occur on the exposed faces before removal from the mold.

After microfabrication of building blocks and functionalization with chemically distinct surfaces, the relevant “click” precursor groups are grafted to the surface of substrates and/or feedstock to produce surfaces with the desired functionality. The specificity of click reactions will potentially enable multiple reactions to be performed simultaneously, providing maximum versatility in the design of the final assembly process. In some embodiments, all “click” reactions may be performed under conditions where they are spontaneous, such that when two surfaces come into contact they react instantly to form a strong, permanent bond. In other embodiments, for example, if the fast rate of reaction leads to an unacceptable level of defects, the reactions may be performed under activated conditions, where the addition of a catalyst (Cu for azide-alkyne, a thiol reductant for thiol-maleimide, aniline for oxime chemistry, or the oxidant for phenol oxidative coupling) is used to trigger the covalent bond only once the particles have annealed into the correct configuration. In this case, weak non-covalent interactions such as hydrogen bonding donors/acceptors or electrostatic interactions can be used to promote appropriate orientation of feedstock on a substrate or other feedstock before covalent bond formation.

In some embodiments, a linker may be used to join the click chemical groups to metal patterns on a substrate and/or to feedstock elements. The linker may be considered a spacer between the surface functionalization (i.e. thiol) and the click chemistry. Examples of linkers include alkyls, aryls, or heteroatom substituted alkyl chains (which allow tunability of solubility, spacing, and/or mechanical stiffness.

### DNA Selective Assembly

In the field of medical diagnostics, DNA selective sensors have been developed that allow for one to detect the presence of one or more pathogens (for example, virus or bacteria) in a fluid sample by sensing the presence of strands of DNA specific to the one or more pathogens. Various DNA selective sensors include a sensor element, for example, a thin gold wire or other nanostructure to which a portion of a strand of DNA complimentary to the DNA of a pathogen of interest has been attached. When a strand of DNA of the pathogen having an order of base units (A, C, G, T) complimentary to the strand of DNA attached to the sensor element contacts the strand of DNA attached to the sensor element, the two strands of DNA bond together and produce a mechanical or electrical change on the sensor element that may be detected to provide an indication of the presence of the pathogen.

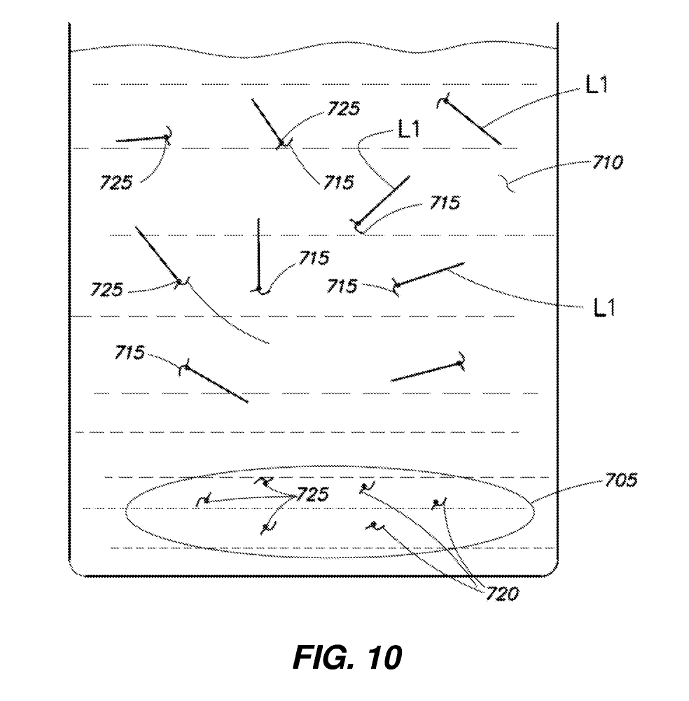

In some embodiments, the ability of complimentary strands of DNA to selectively bond to one another may be capitalized on to provide for a method of joining micro-scale or nano-scale feedstock elements as disclosed herein. For example, in some embodiments, a first strand of DNA is bonded to a substrate in locations where it is desired to attach first micro-scale feedstock elements. A strand of DNA complimentary to the first strand of DNA is bonded to an area of a first micro-scale or nano-scale feedstock elements desired to be bonded to the substrate. As illustrated in FIG. 10, the first micro-scale or nano-scale feedstock elements L1 are placed in a solution 710 and the substrate 705 is exposed to the solution 710. The first micro-scale or nano-scale feedstock elements L1 are then aligned with and positioned on the substrate 705 via a DFA process, for example, using dielectrophoresis as described above. When a strand of DNA 715 on one of the first micro-scale or nano-scale feedstock elements L1 comes into proximity with a complimentary strand of DNA 720 on the substrate 705, the two strands of DNA are drawn together, joining the first micro-scale or nano-scale feedstock element L1 to the substrate 705.

In some embodiments, in addition to providing for bonding of the first micro-scale or nano-scale feedstock element L1 to the substrate 705 with the complimentary DNA strands, additional bonding mechanisms 725 are provided. For example, in addition to the complementary DNA strands, one or both of the substrate 705 and the first micro-scale or nano-scale feedstock element L1 are provided with an additional bonding mechanism 725 at the desired bonding locations. The additional bonding mechanisms 725 may include, for example, but without limitation, an adhesive that may be activated by heat (wax, hot-melt adhesive, etc.) or exposure to one or more forms of radiation (UV light, actinic radiation, etc.) and/or a solder material (for example, an indium/gold or lead/tin eutectic alloy). After the first micro-scale or nano-scale feedstock element L1 is bonded to the substrate 705 via the complimentary DNA strands, the additional bonding mechanisms may be activated by application of heat or radiation to form a bond between the first micro-scale feedstock element L1 and the substrate 705 that may be stronger than the bond between the complimentary DNA strands and that may be more robust in dry environments than the bond between the complimentary DNA strands.

Additional micro-scale or nano-scale feedstock elements may be functionalized with DNA strands complimentary to other DNA strands bonded to desired areas on the first micro-scale or nano-scale feedstock element L1 to provide for the additional micro-scale feedstock elements to be bonded to the first micro-scale or nano-scale feedstock element L1 in a similar manner as the first micro-scale or nano-scale feedstock element L1 is bonded to the substrate 705. This DNA assisted bonding process may be extended to join a plurality of levels of micro-scale or nano-scale feedstock elements into a desired structure.

Three Dimensional Lattice Structures Formed from Nano-Scale Feedstock

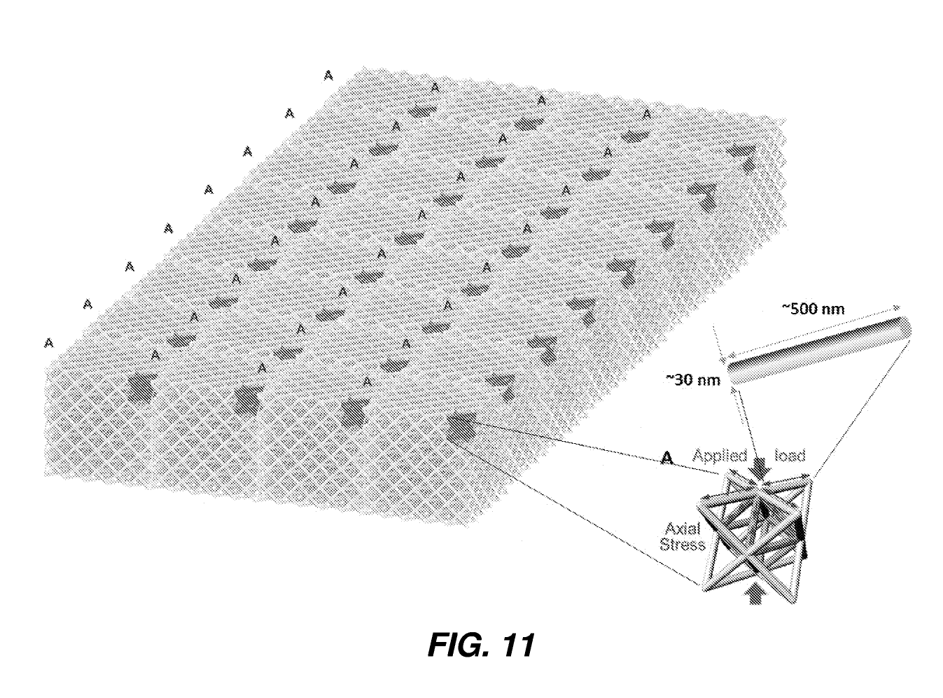

Micro and nano-scale feedstock elements may be joined with methods disclosed herein to form meta-materials including lattice structures that have properties, for example, strength to weight ratios, weight specific energy absorption, stiffness, strength to density ratios, and other physical or optical, electrical, or magnetic properties that are unobtainable with conventional engineering materials. In the specific example of a structural aerogel, a meta-material aerogel may be formed from 30 nm diameter×500 nm long SiO₂ struts fabricated in accordance with methods disclosed herein and assembled into an octet truss structure using bonding methods disclosed herein. See FIG. 11. The calculated physical and mechanical properties of this meta-material aerogel as compared to those of a conventional silica aerogel are illustrated in Table 1 below:

TABLE 1

| | Silica | Meta-Material |
|---|---|---|
| | Aerogel | Aerogel |
| Strut radius (nm) | | 30 |
| Strut length (nm) | | 500 |
| Solid volume fraction | ~5% | 2.4% |
| Solid density (kg/m³) | ~100 | 63.6 |
| Solid density (g/cm³) | ~0.1 | 0.064 |
| Young's modulus (Pa) | 1-10 × 10⁶ | 93.3 × 10⁶ |
| Yield stress (buckling of struts) (Pa) | 16 × 10³ | 240.9 × 10⁶ |
| Internal surface area (m²/g) | 600 - 1,000 | 1.23 |

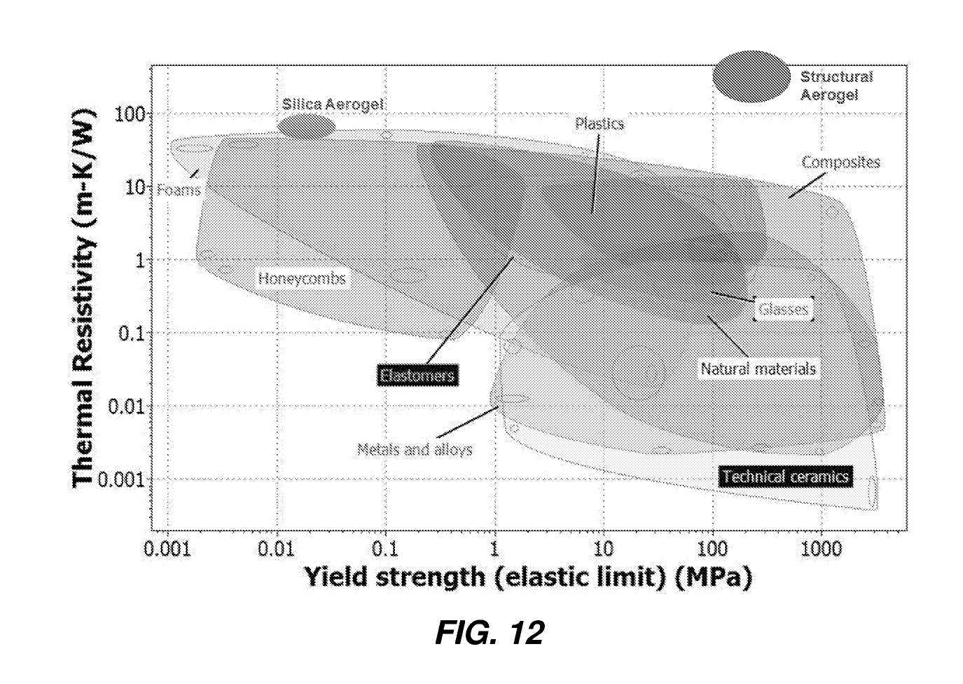

A structural aerogel may also exhibit greater insulative behavior (lesser thermal conductivity) that a conventional silica aerogel because the octet truss structure presents a higher thermal resistance than the random network of a conventional silica aerogel. The structural aerogel may this exhibit a unique combination of thermal insulation and mechanical strength as compared to conventional engineering materials (See FIG. 12). These properties would make the structural aerogel a desirable material for use as an insulator in, for example, aircraft and spacecraft.

Other lattice structures formed from micro or nano-scale feedstock elements may provide for more than a ten times improvement in weight specific energy absorption and may promise greatly reduced transmitted impulse and contact stress as compared to conventional foams, elastomers, or honeycomb structures. These improvements in dynamic energy absorption of lattice structures formed from micro or nano-scale feedstock elements are due to tailorable lattice architecture (e.g., selection of materials, topology, and layering), greatly superior lattice strut lengths, and the ability to greatly reduce relative density, thereby delaying the onset of “densification” under stress. Lattice structures formed from micro or nano-scale feedstock elements may thus be desirable in implementations including, for example, personal protective equipment (sporting equipment such as helmets or military armor), vehicle crash protection (either in impact surfaces or in seats to act as velocity change buffers to reduce spinal injuries), air blast protection for personnel, vehicles, and structures, and underwater impulse/shock protection for personnel and equipment.

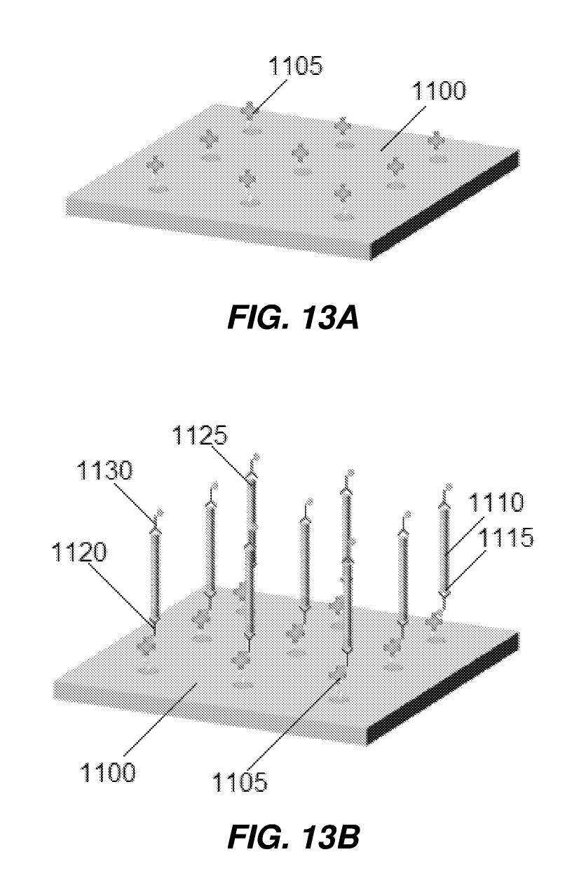

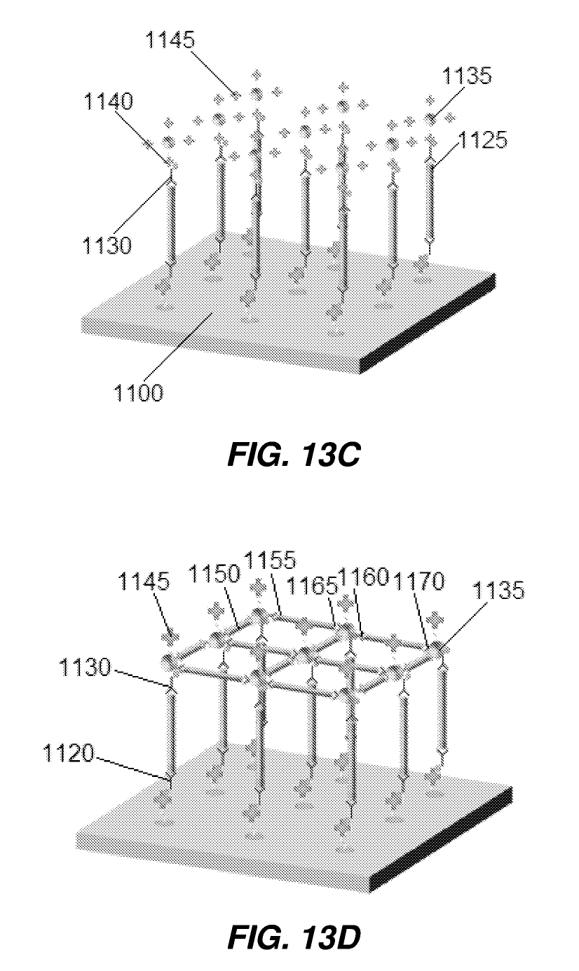

An example of a method of forming a three dimensional lattice from micro or nano-scale feedstock elements is illustrated in FIGS. 13A-13D. In this example, the bonding between different elements is described as being performed via complimentary click chemical groups, however, it is to be understood that complimentary DNA strands on the different elements may additionally or alternatively be utilized to bond different elements.

FIG. 13A illustrates a substrate 1100 that is functionalized with click chemical groups 1105 in a defined pattern. For example, the substrate may be patterned with an array of gold dots to which the first side, for example, a thiol side of a bifunctional click chemical molecule may be bonded to or may include a pattern of exposed silicon islands to which a silane side of a bifunctional click chemical molecule may be bonded to. A second side of the bifunctional click chemical molecule may include a click chemical group 1105, for example, an azide or one of the other click chemical groups discussed above. In other embodiments both the first and second sides of the click chemical molecule may be functionalized with the same species of click chemical group. In some embodiments, the click chemical molecules may include a click chemical group or groups connected to long-chain molecules (for example, polyethylene glycol (PEG), a polymer chain, or a peptide chain). The long-chain molecules may provide flexibility to the click chemical molecules and/or the ability of the different ends of the click chemical molecules to orient in different directions.

In some embodiments, the substrate 1110 is conductive (for example, highly doped silicon) and/or is coated with a conductive thin film, for example, a metal, indium tin oxide, or another conductive material to facilitate the spread of charge across the surface of the substrate 1110 to aid in creating an electric field to align, orient, and/or move micro-scale feedstock elements into place on the surface of the substrate 1110.

The substrate is exposed to a fluid including a first set of feedstock elements 1110, for example, micro or nano-scale rods or bars having first ends 1115 functionalized with click chemical groups 1120 complimentary to the click chemical groups 1105 on the substrate 1100. For example, the first ends of 1115 of the first set of feedstock elements 1110 may be coated with SiO₂ (or the first set of feedstock elements 1110 may be formed from SiO₂) to which the silane side of a bifunctional click chemical molecule having an alkyne group on a second side of the molecule has been bonded. The bifunctional click chemical molecule (and/or click chemical molecules bonded to the substrate, linker elements, or any of the other feedstock elements disclosed herein) may also include an intermediate chemical group, for example, PEG, a polymer chain, or a peptide chain bonded between the first and second click chemical groups (e.g., the silane and alkyne click chemical groups). The micro or nano-scale rods or bars have second ends 1125 that are functionalized with either the same click chemical groups 1120 as the first ends 1115, the same click chemical groups 1105 as the substrate 1100, or different click chemical groups 1130. For example, the second ends 1125 of the micro or nano-scale rods or bars may be coated with gold to which the thiol side of a bifunctional click chemical molecule having a second side including an azide is bonded to. Alternatively, the second ends 1125 of the micro or nano-scale rods or bars may be coated with or be formed from SiO₂ to which the silane side of a bifunctional click chemical molecule having a second side including an alkyne group is bonded to.

The first set of feedstock elements 1110 are aligned on the substrate 1100 using one of the directed fluid assembly methods disclosed above or are permitted to diffuse through the fluid until the click chemical groups 1120 on the first ends 1115 come into contact with the click chemical groups 1105 on the click chemical molecules bonded to the substrate 1100. For example, the free ends of the click chemical molecules may be subject to forces caused by Brownian motion of the fluid in which the substrate is immersed and may move until coming into contact with the click chemical groups 1120 on the first ends 1115 of the first set of feedstock elements 1110. The click chemical groups 1105 and 1120 bond to one another, bonding the first set of feedstock elements 1110 to the substrate 1100 in a defined pattern and orientation. In some embodiments, different bonding points on the substrate 1100 include different click chemical groups and different ones of the first set of feedstock elements 1110 may have different complimentary click chemical groups and may include different materials or have different sizes or shapes than others of the first set of feedstock elements 1110. Different ones of the first set of feedstock elements 1110 having different properties may bond to different predetermined spots on the substrate 1100.

Functionalized areas of the substrate 1100 may have areas greater than areas of the portions of feedstock elements 1110 (for example, areas of end portions of the feedstock elements) that bond to the functionalized areas of the substrate. Accordingly, multiple feedstock elements may bond to individual functionalized areas of the substrate.

The second ends 1125 of the first set of feedstock elements 1110 are then exposed to linker elements 1135, for example, by immersing the substrate 1100 including the bonded first set of feedstock elements 1110 in another fluid including the linker elements 1135. The linker elements 1135 include at least one portion or side functionalized with a click chemical group 1140 complimentary to the click chemical group 1130 on the second ends 1125 of the first set of feedstock elements 1110. The linker elements 1135 may be spheres or three dimensional prisms with a desired number of sides, for example, cubes. In some embodiments, the linker elements 1135 may include a click chemical group or groups connected to long-chain molecules (for example, polyethylene glycol, a polymer chain, or a peptide chain). Chemical groups on first ends of the long chain molecules may bond to bodies of the linker elements 1135 while click chemical groups bonded to another side or portion of the long chain molecules may include the click chemical group 1140 complimentary to the click chemical group 1130 on the second ends 1125 of the first set of feedstock elements 1110. The long-chain molecules may provide flexibility to the linker elements 1135 and/or the ability for click chemical groups on different sides or portions of the linker elements 1135 to orient in different directions. Different sides or portions of the linker elements 1135 (or associated long-chain molecules) may be functionalized with the same or different click chemical groups 1140, 1145, which, in some embodiments, may be the same click chemical groups 1105 that are bonded to the substrate 1100 or the first or second ends of the first set of feedstock elements 1110.

The linker elements 1135 maybe sized and/or shaped to set the coordination and/or relative angle between different feedstock elements, or groups thereof, that bond to the linker elements 1135. For example, for the linker elements 1135 illustrated in FIG. 13C, it may be desirable to size the linker elements (illustrated as spheres in this example) to be sized so that six feedstock element rods would fit on them to provide the desired lattice structure as illustrated in FIG. 13D to be formed. If the linker elements 1135 were too large then extra, unneeded, and possibly undesired feedstock element rods may bond to the linker elements 1135. If the linker elements 1135 were too small then an insufficient number of feedstock element rods may be able to attach to the linker elements 1135 to provide for the desired lattice structure to be formed. By methods including both properly setting the coordination of the linker elements 1135 and the lengths of linking feedstock element rods, a desired type of lattice or truss structure may be fabricated.

In some embodiments, the strength of bonds between linker elements 1135 and feedstock elements may be controlled and bonds between certain feedstock element types and/or bonds to certain sides of the linker elements 1135 may have different strengths. For example, certain bonds may bend easily (for example, to simulate a pin joint between elements) and other bonds may be more rigid. The different bond strengths can be tuned by tuning the length or materials or other properties of long-chain molecules disposed between click chemical groups in click chemical molecules bonded to different sides of the linker elements 1135 and/or to different linker elements 1135 and/or to different feedstock elements. In some embodiments long-chain molecules may be disposed between click chemical groups in click chemical molecules bonded to only selected different sides of the linker elements 1135 and/or to different linker elements 1135 and/or to different feedstock elements.

The linker elements 1135 are brought into contact with the second ends 1125 of the first set of feedstock elements 1110 using one of the directed fluid assembly methods disclosed above or through diffusion or under the influence of Brownian motion of the molecules of the fluid in which the substrate is immersed, and the click chemical groups 1140 of the linker elements 1135 bond to the click chemical groups 1130 on the second ends 1125 of the first set of feedstock elements 1110, bonding the linker elements 1135 to the second ends 1125 of the first set of feedstock elements 1110. In some embodiments, different linker elements 1135 having different click chemical groups and/or different shapes or other properties may bond to different ones of the first set of feedstock elements 1110.

The free sides of the linker elements 1135 are then exposed to a second set of feedstock elements 1150, for example, by immersing the substrate 1100 including the bonded first set of feedstock elements 1110 and linker elements 1135 in another fluid including the second set of feedstock elements 1150. The second set of feedstock elements 1150 may be rods or bars made of the same or different material(s) than the first set of feedstock elements 1110. The second set of feedstock elements 1150 include first ends 1155 functionalized with a click chemical group 1160 and second ends 1165 functionalized with a click chemical group 1170. Click chemical groups 1160 and 1170 are, in some embodiments, the same chemical groups and may be the same or different from the click chemical groups 1120, 1130 bonded to the first and second ends of the first set of feedstock elements 1110. The click chemical groups 1160, 1170 of the first and second ends 1155, 1165 of the second set of feedstock elements 1150 are complimentary to the click chemical groups 1140, 1145 on free sides of the linker elements 1135. In some embodiments, the click chemical groups 1160, 1170 are bonded to the first and second ends 1155, 1165 of the second set of feedstock elements 1150 via one or more long chain molecules, for example, polyethylene glycol or a polymer chain or chains.

The first and second ends 1155, 1165 of the second set of feedstock elements 1150 are brought into contact with the linker elements 1135 using one of the directed fluid assembly methods disclosed above or through diffusion and the click chemical groups 1140, 1145 of the linker elements 1135 bond to the click chemical groups 1160, 1170 on the first and second ends 1155, 1165 of the second set of feedstock elements 1150, bonding the first and second ends 1155, 1165 of the second set of feedstock elements 1150 to the linker elements 1135 and by extension, to the first set of feedstock elements 1110. Different ones of the second set of feedstock elements 1150 may have different properties, for example, material(s) and/or shapes and/or sizes and may include different click chemical groups and may bond to different of the linker elements 1135 and different feedstock elements in the first set of feedstock elements 1110.

The second set of feedstock elements 1150 may be bonded to the first set of feedstock elements 1110 via the linker elements 1135 such that the longitudinal axes of the second set of feedstock elements 1150 are substantially perpendicular to the longitudinal axes of the first set of feedstock elements 1110. The first and second feedstock elements 1110, 1150 may be bonded together in a lattice structure. The first and second feedstock elements 1110, 1150 may be bonded together at different angles depending on the type of lattice or truss structure desired to be formed. The angle of bonding between the first and second feedstock elements 1110, 1150 may be determined by an angle of end faces of the first and second feedstock elements 1110, 1150 and/or the orientation of click chemical groups on ends of the first and/or second feedstock elements 1110, 1150 and/or by the shape of the linker elements 1135 and/or orientation of click chemical groups on the linker elements 1135. Bonding angles between different click chemical groups may also influence the orientation of the bond between the first and second feedstock elements 1110, 1150. Multiple feedstock elements of the second set of feedstock elements 1150 may be bonded in this manner to individual ends of individual ones of the first set of feedstock elements 1110. The bonded pattern of first and second feedstock elements 1110, 1150 may be considered a mesostructure pattern, a pattern that has characteristic dimensions, for example, dimensions of repeating patterns of bonded feedstock elements, in the meso-scale region between nano-scale and micro-scale dimensional regions.

At least one additional side of at least some of the linker elements 1135 are left free and include click chemical groups 1145 to which additional feedstock elements may be joined. The additional sides of the linker elements 1135 may serve the same function as the click chemical groups bonded to the substrate 1100, providing for additional layers of first and second feedstock elements and linker elements to be joined. Additional layers of first and second feedstock elements and linker elements may be subsequently be joined to build a lattice or truss structure having a desired size. For example, multiple layers of feedstock elements may be joined in accordance with the above disclosed method to form an octet truss as illustrated in FIG. 11.

In some embodiments, mesostructured unit cells that would have linker elements, feedstock elements bonded between linker elements, and open click bonds could be assembled as described in the above paragraph then assembled onto a substrate in layers as described above, or assembled into a superstructure without a substrate.

It should be appreciated that in some embodiments, the linker elements 1135 may be omitted and the various feedstock elements may be joined directly to one another to form a lattice or truss structure. Different portions and/or sides of the feedstock elements may be functionalized with different click chemical groups to control the orientation of bonded feedstock elements. Multiple feedstock elements of one set of feedstock elements may bond to individual functionalized portions of individual feedstock elements of a second set of feedstock elements.

The substrate 1100 may not be necessary in all embodiments. In some embodiments, different feedstock elements may be introduced concurrently with one another in solution and may bond together via complimentary click chemical groups (and/or complimentary DNA strands) to form a lattice or truss structure without the feedstock elements needing to be supported on a substrate. A second set of feedstock elements may bond to both first and second ends of a first set of feedstock elements in solution (or to linker elements on the first and second ends of a first set of feedstock elements) to form at least a portion of a three dimensional lattice or truss structure. Additional feedstock elements may bond to other portions of the linker elements of the first or second feedstock elements to build the lattice or truss structure. This process may be repeated until the lattice or truss structure achieves a desired size.

Further, it should be appreciated that bonding sites including click chemical groups may be present not only on end portions of feedstock elements used to form a lattice or truss structure, but may also be present on intermediate portions or sides of the feedstock elements so that feedstock elements may bond to one another not only at their first and second portions on their ends but also at third portions displaced from their ends.

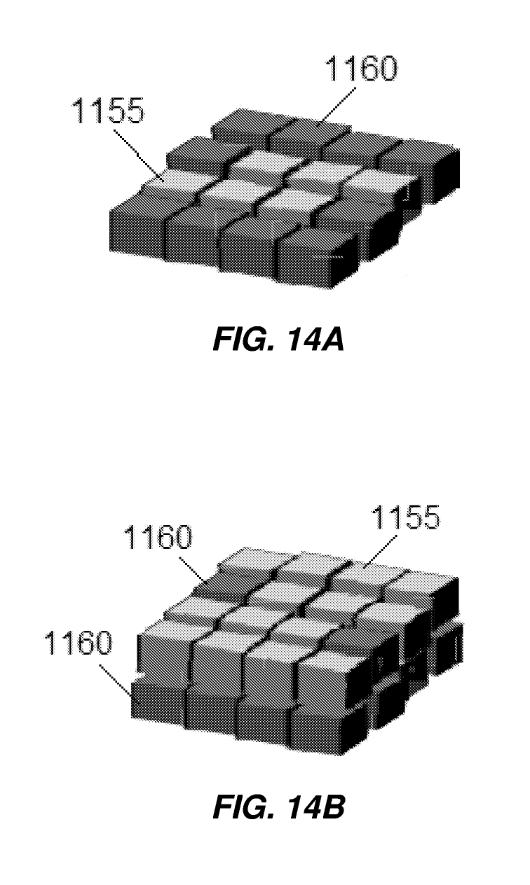

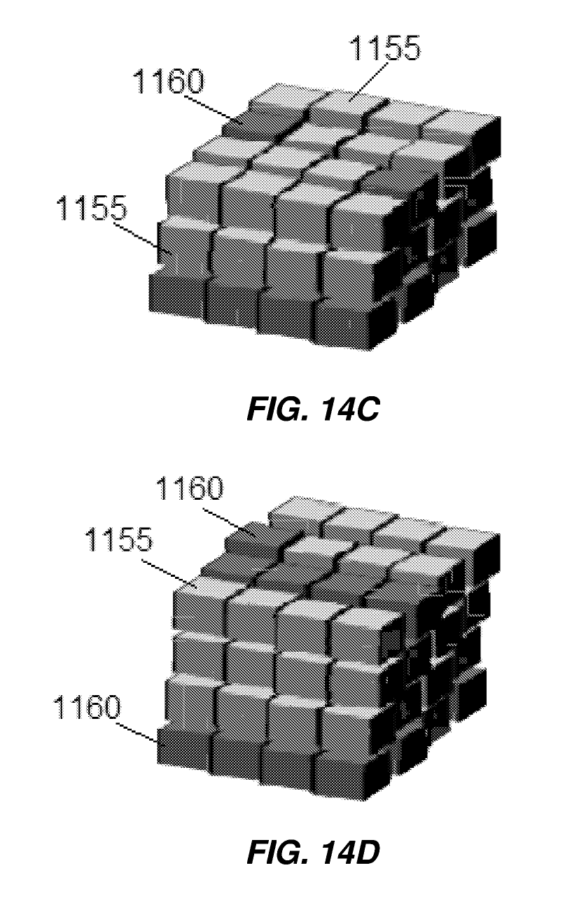

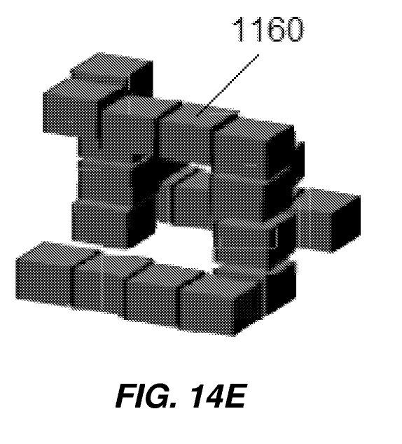

In another aspect, micro or nano-sized prisms may be utilized to form three dimensional structures with useful properties, for example, electronic structures. In one specific example, rectangular prisms, blocks, or cube-shaped feedstock elements fabricated in accordance with methods disclosed herein may be utilized to form an inductor. The inductor is formed from two different types of feedstock elements—feedstock elements comprising or consisting of an insulator, for example SiO₂, and feedstock elements comprising or consisting of a conductive material, for example, copper or aluminum. The feedstock elements may be approximate 1 μm in height, length, and width, although smaller dimensions may be used and the feedstock elements need not necessarily have equal height, length, and width dimensions. The feedstock elements are functionalized with different click chemical groups on different faces to control what other feedstock elements they bond to. As illustrated in FIG. 14A, a first layer of feedstock elements may be joined together to form a first layer of the inductor.

The feedstock elements include insulative feedstock elements 1155 and conductive feedstock elements 1160. In one embodiment, the feedstock elements may be introduced into a fluid medium and moved into contact with one another by one of the directed fluid assembly methods disclosed above or are permitted to diffuse through the fluid until the complimentary click chemical groups on faces of the different feedstock elements contact one another and bond the feedstock elements together. Additional layers of feedstock elements may be added to build up the inductor layer by layer as illustrated in FIGS. 14B-D. The final inductor structure is illustrated in FIG. 14E, where only the conductive feedstock 1160 elements are illustrated for clarity. Other micro- or nano-scale electric or electronic devices, for example, resistor, capacitors, or transistors may be fabricated in a similar manner and even brought into contact with one another to form three dimensional electrical circuits using directed fluid assembly and click chemical bonding as disclosed herein. In some embodiments, the assembled micro- or nano-scale electric or electronic devices may be annealed after assembly utilizing the complimentary click chemicals (or complimentary DNA strands) to, for example, increase bond strength, and/or conductivity between the feedstock elements.

Many different types of lattice or truss structures may be formed from micro or nano-scale feedstock elements as disclosed herein. The types of the lattice or truss structures may be determined based on factors including, for example, the shape and size of the different micro or nano-scale feedstock elements that may be utilized, the orientation of click chemical groups on the micro or nano-scale feedstock elements, the shape and size of the linker elements, and the orientation of different click chemical groups on the linker elements.

One type of lattice that may be formed from micro or nano-scale feedstock elements as described herein is an octet truss structure. An octet truss has a unit cell A as illustrated in FIG. 11. The octet truss structure was first described by Richard Fuller in 1961 in U.S. Pat. No. 2,986,241 for building construction. It includes a face-centered cubic (fcc) lattice with eight tetrahedra distributed on the faces of an octahedral core. Each unit cell or node has 12 struts. Octet truss structures exhibit a stiffness to density ratio that is significantly higher than that of many other lattice or truss structures.

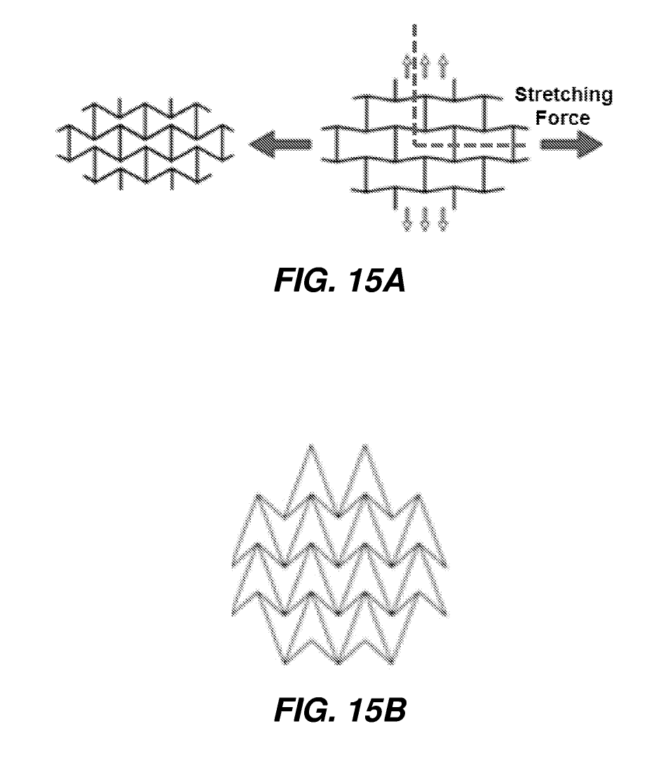

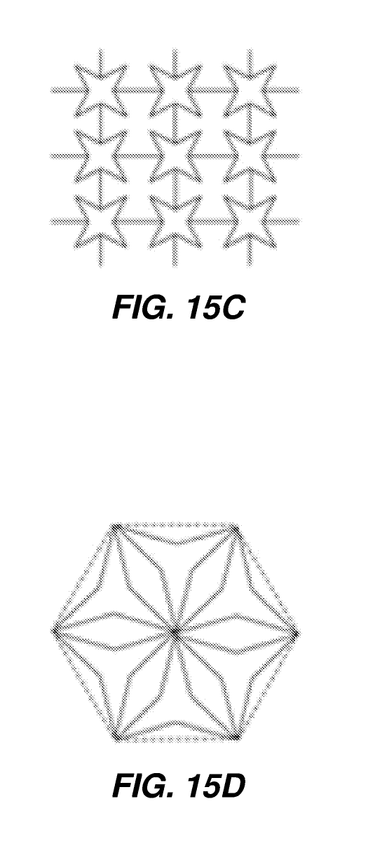

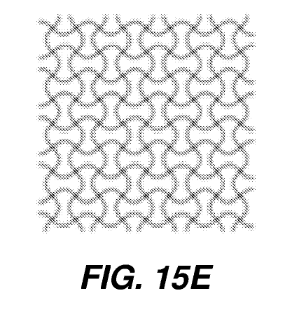

Another type of lattice that may be formed from micro or nano-scale feedstock elements as described herein include auxetic material lattices. Auxetic materials are materials that have a negative Poisson ratio and that expand in a widthwise direction when pulled on and strained in a lengthwise direction as illustrated in FIG. 15A. The structure illustrated in FIG. 15A is classified as a re-entrant structure. Different re-entrant auxetic material lattice structures that may be formed from micro or nano-scale feedstock elements as described herein are illustrated in FIGS. 15A-15E. These structures are illustrated in two dimensional form for ease of illustration.

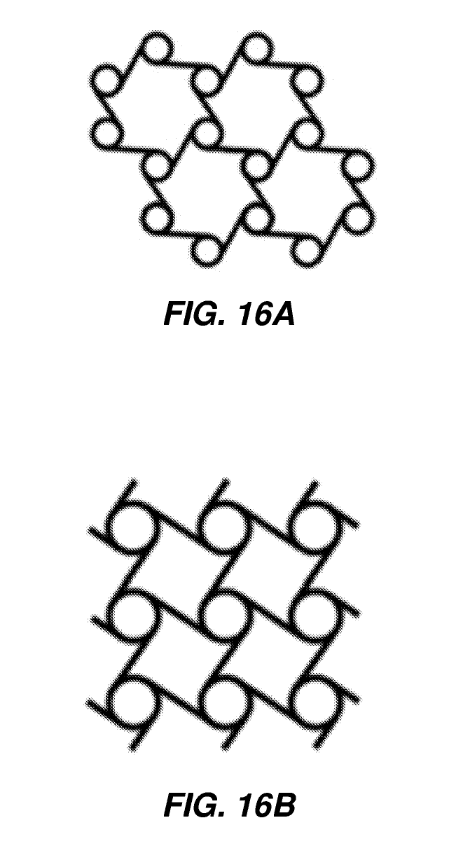

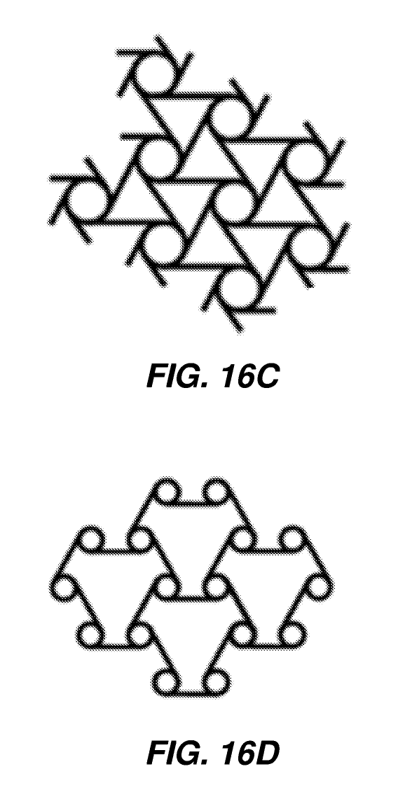

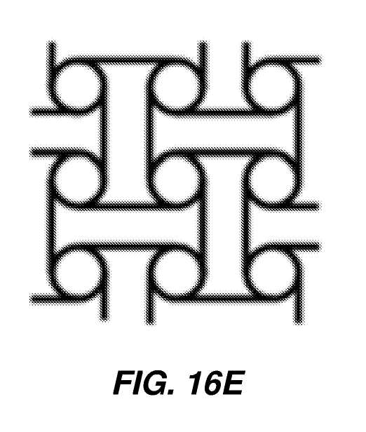

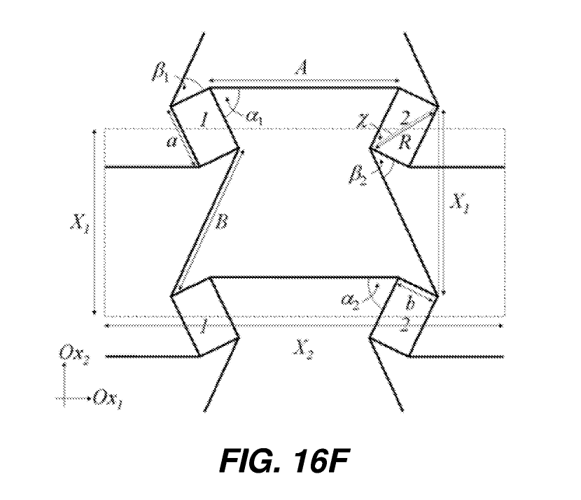

Auxetic material lattices that may be formed from micro or nano-scale feedstock elements as described herein may include chiral material lattices. Chiral units in chiral structures include ligaments that are attached to nodes with rotational symmetry. The structures may be left handed or right handed. If the nodes are on opposite sides of the ligaments, the structure is considered chiral. If the nodes are on the same sides of the ligaments the structures is considered anti-chiral (racemic). “Meta-chiral” structures are those which include ligaments attached to nodes but with degrees of rotational symmetry that are different from the number of ligaments attached to each node. An example of a trichiral structure is illustrated in FIG. 16A. An example of a tetrachiral structure is illustrated in FIG. 16B. An example of a hexachiral structure is illustrated in FIG. 16C. An example of an anti-trichiral structure is illustrated in FIG. 16D. An example of an anti-tetrachiral structure is illustrated in FIG. 16E. These structures are illustrated in two dimensional form for ease of illustration. For ease of fabrication, the circular portions of the chiral material lattices disclosed herein may be replaced by rectangles or other polygons, for example, as illustrated in FIG. 16F.

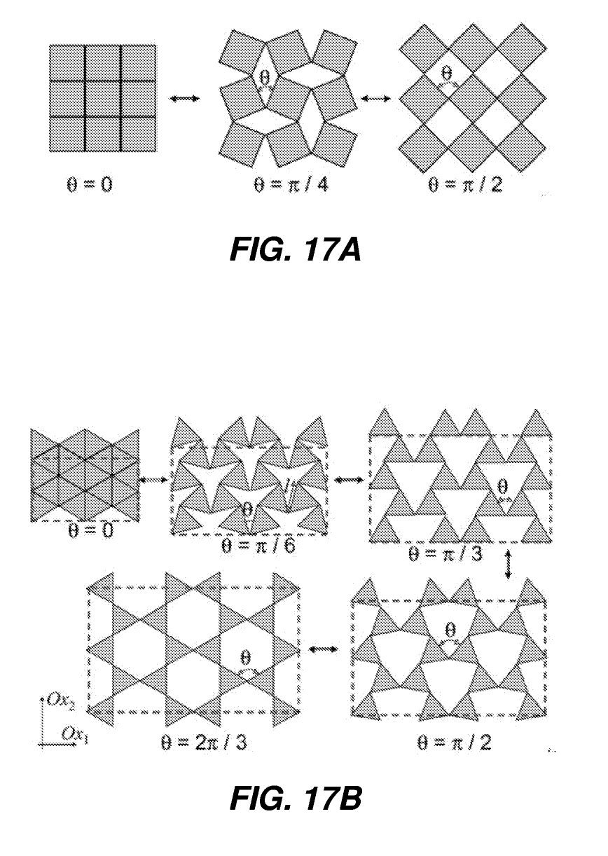

Auxetic material lattices that may be formed from micro or nano-scale feedstock elements as described herein may include rotating units. Micro or nano-scale units may be joined with rotating bonds such that the units rotate with applied strain as illustrated in FIGS. 17A and 17B.

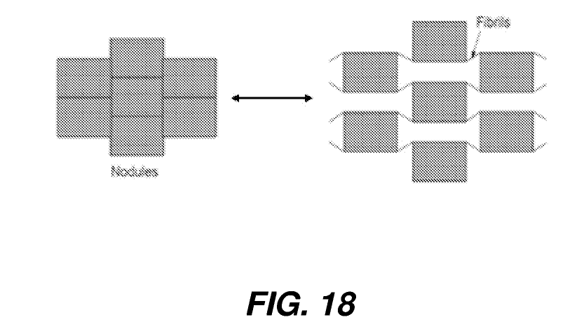

Further auxetic structures that may be formed from micro or nano-scale feedstock elements as described herein may include nodules joined by fibrils. As illustrated in FIG. 18 as the nodules are pulled apart in a lengthwise direction, the fibrils pull the nodules apart from one another in a widthwise direction.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A method of assembly of micro/nano-scale objects into a three dimensional structure, the method comprising:
forming a pattern of a first functional moiety on a surface of a substrate;
contacting the surface of the substrate with a first liquid suspension including first micro/nano-scale feedstock elements functionalized with a second functional moiety, complimentary to the first functional moiety, on first portions of the first micro/nano-scale feedstock elements and functionalized with a third functional moiety on second portions of the first micro/nano-scale feedstock elements;
aligning the first portions of the first micro/nano-scale feedstock elements in the first liquid suspension with the surface of the substrate;
facilitating bonding the second functional moieties to the first functional moieties to form a first mesostructure pattern of the first micro/nano-scale feedstock elements on the surface of the substrate;
contacting the first mesostructure pattern of the first micro/nano-scale feedstock elements on the surface of the substrate with a second liquid suspension including micro/nano-scale linker feedstock elements functionalized with a fourth functional moiety, complimentary to the third functional moiety, on first portions of the micro/nano-scale linker feedstock elements and functionalized with a fifth functional moiety on second portions of the micro/nano-scale linker feedstock elements;
aligning the first portions of the micro/nano-scale linker feedstock elements in the second liquid suspension with the second portions of a first group of the first micro/nano-scale feedstock elements;
facilitating bonding the fourth functional moieties to the third functional moieties to form a second mesostructure of micro/nano-scale objects on the surface of the substrate;
contacting the second mesostructure pattern of micro/nano-scale feedstock elements on the surface of the substrate with a third liquid suspension including second micro/nano-scale feedstock elements functionalized with a sixth functional moiety, complimentary to the fifth functional moiety, on first portions of the second micro/nano-scale feedstock elements and functionalized with a seventh functional moiety, on second portions of the second micro/nano-scale feedstock elements;
aligning the first portions of the second micro/nano-scale feedstock elements in the third liquid suspension with the second portions of a first group of the micro/nano-scale linker feedstock elements;
aligning the second portions of the second micro/nano-scale feedstock elements in the third liquid suspension with the second portions of a second group of the micro/nano-scale linker feedstock elements; and
facilitating bonding the sixth and seventh functional moieties to the fifth functional moieties to form the three-dimensional structure of micro/nano-scale objects on the surface of the substrate.

2. The method of claim 1, wherein facilitating bonding the fourth functional moieties to the third functional moieties and facilitating bonding the sixth and seventh functional moieties to the fifth functional moieties includes leaving some of at least one of the fourth, fifth, sixth, or seventh functional moieties unbonded.

3. The method of claim 1, further comprising:
contacting the three dimensional structure of micro/nano-scale objects with a third liquid suspension including third micro/nano-scale feedstock elements;
aligning and positioning first portions of the third micro/nano-scale feedstock elements in the third liquid suspension with third portions of the second micro/nano-scale feedstock elements; and
facilitating bonding the first portions of third micro/nano-scale feedstock elements to the third portions of the second micro/nano-scale feedstock elements.

4. The method of claim 3, wherein portions of micro/nano-scale feedstock elements are bonded to portions of other micro/nano-scale feedstock elements with complimentary click chemical groups.

5. The method of claim 3, wherein portions of micro/nano-scale feedstock elements are bonded to portions of other micro/nano-scale feedstock elements with complimentary DNA strands.

6. The method of claim 3, wherein aligning and positioning portions of micro/nano-scale feedstock elements with portions of other micro/nano-scale feedstock elements includes aligning and positioning the portions of the micro/nano-scale feedstock elements with the portions of the other micro/nano-scale feedstock elements with an electric field to create electrophoretic and/or dielectrophoretic forces.

7. The method of claim 3, wherein aligning and positioning portions of micro/nano-scale feedstock elements with portions of other micro/nano-scale feedstock elements includes aligning and positioning the portions of the micro/nano-scale feedstock elements with the portions of the other micro/nano-scale feedstock elements utilizing flow of fluid in the third liquid suspension.

8. The method of claim 3, wherein aligning and positioning portions of micro/nano-scale feedstock elements with portions of other micro/nano-scale feedstock elements includes aligning and positioning the portions of the micro/nano-scale feedstock elements with the portions of the other micro/nano-scale feedstock elements utilizing a magnetic field.

9. The method of claim 3, wherein aligning and positioning portions of micro/nano-scale feedstock elements with portions of other micro/nano-scale feedstock elements includes aligning and positioning the portions of the micro/nano-scale feedstock elements with the portions of the other micro/nano-scale feedstock elements utilizing optical trapping.

10. The method of claim 3, wherein one or more of the first, second, or third micro/nano-scale feedstock elements include one or more of nanotubes, nanorods, or nanoparticles.

11. The method of claim 10, wherein the one or more of nanotubes, nanorods, and nanoparticles comprise one of carbon nanotubes, nanorods, and nanoparticles, boron nanotubes, nanorods, and nanoparticles, or combinations thereof.

12. The method of claim 10, wherein the one or more of nanotubes, nanorods, and nanoparticles are bonded to portions of other micro/nano-scale feedstock elements with complimentary click chemical groups.

13. The method of claim 10, wherein the one or more of nanotubes, nanorods, and nanoparticles are bonded to portions of other micro/nano-scale feedstock elements with complimentary DNA strands.

14. The method of claim 10, wherein aligning and positioning the portions of the one or more of carbon nanotubes, nanorods, and nanoparticles with portions of other micro/nano-scale feedstock elements includes aligning and positioning the portions of the one or more of nanotubes, nanorods, and nanoparticles with the portions of the other micro/nano-scale feedstock elements with an electric field to create electrophoretic and/or dielectrophoretic forces

15. The method of claim 10, comprising concurrently bonding at least two of i) the first portions of the first micro/nano-scale feedstock elements to the substrate, ii) the second portions of the first group of the first micro/nano-scale feedstock elements to the first portions of the second micro/nano-scale feedstock elements, iii) the second portions of the second group of the first micro/nano-scale feedstock elements to the second portions of the second micro/nano-scale feedstock elements, iv) the first portions of the third micro/nano-scale feedstock elements to the third portions of the second micro/nano-scale feedstock elements, and v) the one or more of nanotubes, nanorods, and nanoparticles to portions of other micro/nano-scale feedstock elements.

16. The method of claim 1, wherein the third functional moiety is the same as the first functional moiety.

17. The method of claim 16, wherein the fourth functional moiety is the same as the second functional moiety.

18. The method of claim 1, wherein the third functional moiety is the same as the second functional moiety.

19. The method of claim 18, wherein the fourth functional moiety is the same as the first functional moiety.

20. The method of claim 1, wherein facilitating bonding between complimentary functional moieties includes initiating bonding between the complimentary functional moieties by one of application of thermal energy to the complimentary functional moieties, application of radiation to the complimentary functional moieties, exposing the complimentary functional moieties to a chemical catalyst and/or by changing a pH of a fluid suspension in which the complimentary functional moieties are immersed.

21. The method of claim 1, further comprising bonding the first functional moiety with a linker molecule to an adhesion element bonded to the surface of the substrate to form the pattern of the first functional moiety on the surface of the substrate.

22. The method of claim 21, wherein the adhesion element comprises one or more of a metal, silicon, and silicon dioxide.

23. The method of claim 1, further comprising bonding functional moieties with linker molecules to adhesion elements bonded to portions of micro/nano-scale feedstock elements.

24. The method of claim 1, further comprising facilitating bonding a plurality of micro/nano-scale feedstock elements to portions of single other micro/nano-scale feedstock elements.

25. The method of claim 1, further comprising facilitating bonding a plurality of the first micro/nano-scale feedstock elements to individual bonding sites including the first functional moiety on the surface of a substrate.

26. The method of claim 1, wherein facilitating bonding the second functional moieties to the first functional moieties includes facilitating bonding a first click chemical group to a complimentary click chemical group.

27. The method of claim 1, wherein facilitating bonding the second functional moieties to the first functional moieties includes facilitating bonding a first DNA strand to a complimentary DNA strand.

28. The method of claim 27, further comprising bonding the first micro/nano-scale feedstock elements to the surface of the substrate with an additional bonding mechanism.

29. The method of claim 1, further comprising forming micro/nano-scale feedstock elements by sequential infiltration synthesis of a domain of a block copolymer.

30. The method of claim 1, further comprising forming micro/nano-scale feedstock elements by a method comprising:
depositing a liquid phase block copolymer into an area defined on or in an upper layer of a multi-layer substrate;
annealing the block copolymer to facilitate separation of the block copolymer into multiple aligned polymer domains;
removing one of the polymer domains;
etching through a remaining polymer domain and into the upper layer of the multi-layer substrate; and
obtaining micro/nano-scale feedstock elements by separating etched portions of the upper layer of the multi-layer substrate from a second layer of the multi-layer substrate.

31. The method of claim 1, further comprising forming micro/nano-scale feedstock elements by a method comprising:
depositing a liquid phase block copolymer into an area defined on or in an upper layer of a multi-layer substrate;
annealing the block copolymer to facilitate separation of the block copolymer into multiple aligned polymer domains;
converting one of the polymer domains into an inorganic material using sequential infiltration synthesis;
etching through a second of the polymer domains and into the upper layer of the multi-layer substrate using the inorganic material as an etch mask; and
obtaining the micro/nano-scale feedstock elements by separating etched portions of the upper layer of the multi-layer substrate from a second layer of the multi-layer substrate.

32. The method of claim 31, further comprising, prior to separating the etched portions of the upper layer of the multi-layer substrate from the second layer of the multi-layer substrate:
depositing and patterning a layer of photoresist on the micro/nano-scale feedstock elements, patterning of the layer of photoresist exposing portions of the micro/nano-scale feedstock elements;
defining lengths of the micro/nano-scale feedstock elements by etching through exposed portions of the micro/nano-scale feedstock elements;
functionalizing exposed end portions of the micro/nano-scale feedstock elements while the micro/nano-scale feedstock elements are embedded in the photoresist; and
removing the photoresist.

33. The method of claim 29, wherein forming micro/nano-scale feedstock elements includes forming micro/nano-scale feedstock elements with at least one dimension between about 5 nm and about 50 nm.

34. The method of claim 1, further comprising functionalizing micro/nano-scale feedstock elements with functional moieties by a method including:
depositing a first bonding material on portions of the micro/nano-scale feedstock elements; and
exposing the first bonding material to a multifunctional click chemical including a chemical group having an affinity for the first bonding material.

35. The method of claim 34, wherein depositing the first bonding material on the portions of the micro/nano-scale feedstock elements includes depositing one of gold, silicon, and silicon dioxide on the portions of the micro/nano-scale feedstock elements.

36. The method of claim 34, wherein exposing the first bonding material to the multifunctional click chemical includes exposing the first bonding material to a chemical including the chemical group having the affinity for the first bonding material, an intermediate chemical group bonded to the chemical group having the affinity for the first bonding material and a further chemical group having an affinity for a second bonding material.

37. The method of claim 36, wherein the intermediate chemical group comprises a polymer chain.

38. A method of assembly of micro/nano-scale objects into a three dimensional lattice or truss structure, the method comprising:
forming a first liquid suspension including first micro/nano-scale feedstock elements functionalized with a first functional moiety on first portions of the first micro/nano-scale feedstock elements and linker elements including a second functional moiety, complimentary to the first functional moiety, on first portions of the linker elements and a third functional moiety on second portions of the linker elements;
aligning the first portions of the first micro/nano-scale feedstock elements in the first liquid suspension with the first portions of the linker elements;
facilitating bonding the second functional moieties to the first functional moieties to bond the linker elements to the first portions of the first micro/nano-scale feedstock elements;
contacting the first micro/nano-scale feedstock elements and linker elements with a second liquid suspension including second micro/nano-scale feedstock elements functionalized with a fourth functional moiety, complimentary to the third functional moiety, on first portions of the second micro/nano-scale feedstock elements and on second portions of the second micro/nano-scale feedstock elements;
aligning the first portions of the second micro/nano-scale feedstock elements in the second liquid suspension with the second portions of a first group of the linker elements;
aligning the second portions of the second micro/nano-scale feedstock elements in the second liquid suspension with the second portions of a second group of the linker elements; and
facilitating bonding the fourth functional moieties to the third functional moieties to form the three dimensional lattice or truss structure.

39. The method of claim 38, further comprising:
contacting the three dimensional lattice or truss structure with a third liquid suspension including third micro/nano-scale feedstock elements functionalized with a fifth functional moiety, complimentary to a sixth functional moiety on a third portion of at least a portion of the linker elements, on first portions of the third micro/nano-scale feedstock elements;
aligning the first portions of the third micro/nano-scale feedstock elements in the third liquid suspension with the third portions of the at least a portion of the linker elements; and
facilitating bonding the fifth functional moieties to the sixth functional moieties.

40. The method of claim 39, comprising aligning the first, second, and third micro/nano-scale feedstock elements into an auxetic truss structure.

41. A three dimensional lattice or truss structure of micro/nano-scale objects comprising:
a plurality of first micro/nano-scale feedstock elements having first portions bonded to first portions of linker elements; and
a plurality of second micro/nano-scale feedstock elements having first portions bonded to second portions of the linker elements and second portions bonded to third portions of the linker elements.

42. The structure of claim 41, wherein the first portions of the plurality of first micro/nano-scale feedstock elements are bonded to the first portions of the linker elements with click chemical bonds.

43. The structure of claim 41, wherein at least a portion of one of the first micro/nano-scale feedstock elements and the second micro/nano-scale feedstock elements have length:width aspect ratios of at least about 20:1.

44. The structure of claim 41, further comprising a plurality of the second micro/nano-scale feedstock elements bonded to each first micro/nano-scale feedstock element.

45. The structure of claim 41, further comprising a plurality of third micro/nano-scale feedstock elements having first portions bonded to fourth portions of the linker elements.

46. The structure of claim 45, wherein the first portions of the plurality of third micro/nano-scale feedstock elements are bonded to fourth portions of the linker elements with click chemical bonds.

47. The structure of claim 45, wherein the first, second, and third micro/nano-scale feedstock elements are arranged into an auxetic truss.

48. The structure of claim 41, further comprising a plurality of the third micro/nano-scale feedstock elements bonded to each second micro/nano-scale feedstock element.

49. A method of assembly of micro/nano-scale objects into a three dimensional structure, the method comprising:
forming a pattern of a first functional moiety on a surface of a substrate;
contacting the surface of the substrate with a first liquid suspension including first micro/nano-scale feedstock elements functionalized with a second functional moiety, complimentary to the first functional moiety, on first portions of the first micro/nano-scale feedstock elements and functionalized with a third functional moiety on second portions of the first micro/nano-scale feedstock elements;
aligning the first portions of the first micro/nano-scale feedstock elements in the first liquid suspension with the surface of the substrate;
facilitating bonding the second functional moieties to the first functional moieties to form a first mesostructure pattern of the first micro/nano-scale feedstock elements on the surface of the substrate;
contacting the first mesostructure pattern of the first micro/nano-scale feedstock elements on the surface of the substrate with a second liquid suspension including second micro/nano-scale feedstock elements functionalized with a fourth functional moiety, complimentary to the third functional moiety, on first portions of the second micro/nano-scale feedstock elements and on second portions of the second micro/nano-scale feedstock elements;
aligning the first portions of the second micro/nano-scale feedstock elements in the second liquid suspension with the second portions of a first group of the first micro/nano-scale feedstock elements;
aligning the second portions of the second micro/nano-scale feedstock elements in the second liquid suspension with the second portions of a second group of the first micro/nano-scale feedstock elements; and
facilitating bonding the fourth functional moieties to the third functional moieties to form the three dimensional structure of micro/nano-scale objects on the surface of the substrate.

50. The method of claim 49, wherein facilitating bonding the fourth functional moieties to the third functional moieties includes facilitating bonding the fourth functional moieties to linker elements and facilitating bonding of the linker elements to the third functional moieties.
